(19) United States
(12) Patent Application Publication
(10) Pub. No.: US 2007/0082872 A1
(43) Pub. Date: Apr. 12, 2007

(54) Indole-containing compounds with anti-tubulin and vascular targeting activity

(76) Inventors: Kevin G. Pinney, Woodway, TX (US); Feng Wang, Jefferson, PA (US); Mallinath Hadimani, Waco, TX (US); Maria Del Pilar Mejia, Normal, IL (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/555,352

(22) PCT Filed: May 3, 2004
(86) PCT No.: PCT/US04/13616

§ 371 Date: Oct. 16, 2006

Related U.S. Application Data
(63) Continuation-in-part of application No. 10/070,484, filed on Oct. 11, 2002, now Pat. No. 6,849,656.
(60) Provisional application No. 60/467,681, filed on May 2, 2003.
(60) Provisional application No. 60/154,639, filed on Sep. 17, 1999.

(51) Int. Cl. A61K 31/675; C07F 9/547; A61K 31/404; C07D 209/36

(52) U.S. Cl. 514/80; 514/418; 548/414; 548/484

(57) ABSTRACT

Trimethoxyphenyl substituted indole ligands have been discovered which demonstrate impressive cytotoxicity as well as a remarkable ability to inhibit tubulin assembly. Such compounds as well as related derivatives are excellent clinical candidates for the treatment of cancer in humans. In addition, certain of these ligands, as pro-drugs, may well prove to be tumor selective vascular targeting chemotherapeutic agents or to have vascular targeting activity resulting in the selective prevention and/or destruction of nonmalignant proliferating vasculature.

### FIELD OF THE INVENTION

Trimethoxyphenyl substituted indole ligands have been discovered which demonstrate impressive cytotoxicity as well as a remarkable ability to inhibit tubulin polymerization. Such compounds as well as related derivatives are excellent clinical candidates for the treatment of cancer in humans. In addition, certain of these ligands, as pro-drugs, may well prove to be tumor selective vascular targeting chemotherapeutic agents or to have vascular targeting activity resulting in the selective prevention and/or destruction of nonmalignant proliferating vasculature.

### BACKGROUND OF THE INVENTION

The cytoskeletal protein tubulin is among the most attractive therapeutic drug targets for the treatment of solid tumors. A particularly successful class of chemotherapeutics mediates its anti-tumor effect through a direct binding interaction with tubulin. This clinically promising class of therapeutics, called tubulin binding agents or anti-tubulin agents, exhibit potent tumor cell cytotoxicity by efficiently inhibiting the polymerization of αβ-tubulin heterodimers into the microtubule structures that are required to facilitate mitotic cell division (Hamel, Medicinal Research Reviews, 1996).

Currently, the most widely recognized and clinically useful anti-tubulin agents are the Vinca Alkaloids, such as Vinblastine and Vincristine (Owellen et al, Cancer Res., 1976; Lavielle et al, J. Med. Chem., 1991) along with Taxanes such Taxol (Kingston, J. Nat. Prod., 1990; Schiff et al, Nature, 1979; Swindell et al, J. Cell Biol., 1981). Additionally, natural products such as Rhizoxin (Nakada et al, Tetrahedron Lett., 1993; Boger et al, J. Org. Chem., 1992; Rao, et al, Tetrahedron Lett., 1992; Kobayashi et al, Pure Appl. Chem., 1992; Kobayashi et al, Indian J. Chem., 1993; Rao et al, Tetrahedron Lett., 1993), the Combretastatins (Lin et al, Biochemistry, 1989; Pettit et al, J. Nat. Prod., 1987; Pettit et al, J. Org. Chem., 1985; Pettit et al, Can. J. Chem., 1982; Dorr et al, Invest. New Drugs, 1996), Curacin A (Gerwick et al, J. Org. Chem., 59:1243, 1994), Podophyllotoxin (Hammonds et al, J. Med. Microbiol, 1996; Coretese et al, J. Biol. Chem., 1977), Epothilones A and B (Nicolau et al., Nature, 1997), Dolastatin-10 (Pettit et al, J. Am. Chem. Soc., 1987; Pettit et al, Anti-Cancer Drug Des., 1998), and Welwistatin (Zhang et al, Molecular Pharmacology, 1996), as well as certain synthetic analogues including Phenstatin (Pettit G R et al., J. Med. Chem., 1998), 2-styrylquinazolin-4(3H)-ones (“SQOs”, Jiang et al, J. Med. Chem., 1990), and highly oxygenated derivatives of cis- and trans-stilbene and dihydrostilbene (Cushman et al, J. Med. Chem., 1991) are all known to mediate tumor cytotoxic activity through a mode of action that includes tubulin binding and subsequent inhibition of mitosis.

Normally, during the metaphase of cell mitosis, the nuclear membrane has broken down and tubulin is able to form centrosomes (also called microtubule organizing centers) that facilitate the formation of the microtubule spindle apparatus to which the dividing chromosomes become attached. Subsequent assembly and disassembly of the spindle apparatus mitigates the separation of the daughter chromosomes during anaphase such that each daughter cell contains a full complement of chromosomes. As antiproliferatives or antimitotic agents, tubulin binding agents exploit the relatively rapid mitosis that occurs in proliferating tumor cells. By binding to tubulin and inhibiting the formation of the spindle apparatus in a tumor cell, the Tubulin Binding Agent can cause significant tumor cell cytotoxicity with relatively minor effects on the slowly dividing normal cells of the patient.

The exact nature of tubulin binding site interactions remain largely unknown, and they definitely vary between each class of Tubulin Binding Agent. Photoaffinity labeling and other binding site elucidation techniques have identified three key binding sites on tubulin: 1) the colchicine site (Floyd et al, Biochemistry, 1989; Staretz et al, J. Org. Chem., 1993; Williams et al, J. Biol. Chem., 1985; Wolff et al, Proc. Natl. Acad. Sci. U.S.A., 1991), 2) the vinca alkaloid site (Safa et al, Biochemistry, 1987), and 3) a site on the polymerized microtubule to which taxol binds (Rao et al, J. Natl. Cancer Inst., 1992; Lin et al, Biochemistry, 1989; Sawada et al, Bioconjugate Chem, 1993; Sawada et al, Biochem. Biophys. Res. Commun., 1991; Sawada et al, Biochem. Pharmacol., 1993). An important aspect of this work requires a detailed understanding, at the molecular level, of the “small molecule” binding domain of both the α and β subunits of tubulin. The tertiary structure of the α,β tubulin heterodimer was reported in 1998 by Downing and co-workers at a resolution of 3.7 Å using a technique known as electron crystallography (Nogales et al, Nature, 1998). This brilliant accomplishment culminates decades of work directed toward the elucidation of this structure and should facilitate the identification of small molecule binding sites, such as the colchicine site, using techniques such as photoaffinity and chemical affinity labeling (Chavan et al, Bioconjugate Chem., 1993; Hahn et al, Photochem. Photobiol., 1992).

Further significance is given to new drugs that bind to the colchicine site since it has recently been shown that many tubulin binding agents also demonstrate activity against malignant proliferating tumor vasculature, as opposed to the tumor itself. Antivascular chemotherapy is an emerging area of cancer chemotherapy which centers on the development of drugs that target the proliferation of the vasculature the supports tumor growth. Much of the research in anti-vascular cancer therapy has focused on understanding the process of new blood vessel formation, known as angiogenesis, and identifying anti-angiogenic agents which inhibit the formation of new blood vessels. Angiogenesis is characterized by the proliferation of tumor endothelial cells and generation of new vasculature to support the growth of a tumor. This growth is stimulated by certain growth factors produced by the tumor itself. One of these growth factors, Vascular Endothelial Growth Factor (“VEGF”), is relatively specific towards endothelial cells, by virtue of the restricted and up-regulated expression of its cognate receptor. Various anti-angiogenic strategies have been developed to inhibit this signaling process at one or more steps in the biochemical pathway in order to prevent the growth and establishment of the tumor vasculature. However, anti-angiogenic therapies act slowly and must be chronically administered over a period of months to years in order to produce a desired effect.

Vascular Targeting Agents (“VTAs”) or vascular damaging agents, are a separate class of antivascular chemotherapeutics. In contrast to anti-angiogenic drugs which disrupt the new microvessel formation of developing tumors, VTAs attack solid tumors by selectively targeting the established tumor vasculature and causing extensive shutdown of tumor blood flow. A single dose of a VTA can cause a rapid and selective shutdown of the tumor neovasculature within a period of minutes to hours, leading eventually to tumor necrosis by induction of hypoxia and nutrient depletion. This vascular-mediated cytotoxic mechanism of VTA action is quite divorced from that of anti-angiogenic agents that inhibit the formation of new tumor vascularization, rather than interfering with the existing tumor vasculature. Other agents have been known to disrupt tumor vasculature, but differ in that they also manifest substantial normal tissue toxicity at their maximum tolerated dose. In contrast, genuine VTAs retain their vascular shutdown activity at a fraction of their maximum tolerated dose. It is thought that Tubulin-binding VTAs selectively destabilize the microtubule cytoskeleton of tumor endothelial cells, causing a profound alteration in the shape of the cell which ultimately leads to occlusion of the tumor blood vessel and shutdown of blood flow to the tumor (Kanthou et al, Blood, 2002).

Combretastatin A4 phosphate prodrug (“CA4P) is one of the leading new candidates from among a relatively small collection of known world compounds with vascular targeting activity (U.S. Pat. No. 5,561,122; Chaplin et al, Anticancer Res., 1999; Tozer et al, Cancer Res., 1999; Pettit and Rhodes, Anti-Cancer Drug Des., 1998; Iyer et al, Cancer Res., 1998; Dark et al, Cancer Res., 1997). Its parent phenol compound, Combretastatin A-4 (“CA4”) was discovered by Professor George R. Pettit (Arizona State University) as an isolate from South African bush willow (Combretum caffrum) in the 1970s. CA4 is a potent inhibitor of tubulin assembly and binds to the colchicine site on β-tubulin. Interestingly, CA4 itself does not demonstrate destruction of tumor vasculature, while CA4P is very active in terms of tumor vasculature destruction. Therefore, the phosphate ester portion of CA4P undergoes dephosphorylation to reveal the potent tubulin binder CA4 that destroys the tumor cell through an inhibition of tubulin assembly.

CA4P is currently the lead drug in a group of tubulin-binding VTAs under clinical development. Other tubulin binding VTAs that have been discovered include the Colchicinoid ZD6126 (Davis et al., Cancer Research, 2002 ) and the Combretastatin analog AVE8032 (Lejeune et al, Proceedings of the AACR., 2002).

An aggressive chemotherapeutic strategy for the treatment and maintenance of solid tumor cancers continues to rely on the development of architecturally new and biologically more potent compounds. The present invention addresses this urgent need by providing a structurally novel class of Tubulin Binding Agent compositions with potent antiproliferative activity and tumor cell cytotoxicity. In addition, the present invention provides the important discovery that corresponding prodrug constructs of these agents have selective effects on the tumor vasculature that are independent of any antimitotic effect on the cells of the tumor. These agents are capable of selectively shutting down the flow of blood to a tumor and causing secondary tumor cell death. Thus the present compositions have expanded clinical utility over known tubulin binding agents.

### SUMMARY OF THE INVENTION

The present invention relates to a discovery of indole compounds that result from the judicious combination of a non-tubulin binding molecular template which, when suitably modified with structural features such as hydroxyl moieties and arylalkoxy groups, are found to function as tubulin binding agents capable of inhibiting tubulin assembly and tumor cell proliferation.

In a first general aspect, the present invention provides indole compounds of the following general formula I: 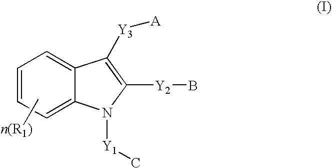
wherein

R₁ is independently selected from OH, nitro, amine, lower alkyl, lower alkoxy, phosphate, or halogen;

n is 0, 1, 2, 3, or 4;

Y₁, Y₂, are Y₃ are optionally a covalent bond, —CO—, —O—, —S—, —CH₂—, or —CH₂O—; and

A, B, and C are alkyl, aryl or H,

wherein at least two of A, B, or C are aryl.

n is desirably 1 or 2. A is desirably a substituted aryl group. In an embodiment, A is trisubstituted; two or three of the substituents may be identical. The substituents may be the same, or different. In a desirable embodiment, the substituents are alkoxy, e.g., methoxy, ethoxy; or hydroxyl. In a preferred embodiment, Y₃ is a carbonyl group.

In another embodiment, A may be a substituted aryl group. In an embodiment, A is di- or tri-substituted; two or three of the substituents may be identical. The substituents may be the same, or different. In a desirable embodiment, the substituents are alkoxy e.g. methoxy, ethoxy; hydroxyl or amino. Preferably Y₃ is a covalent bond in this embodiment.

B is desirably a substituted aryl group. In an embodiment, B is di- or tri-substituted. Two or three of the substituents may be identical. The substituents may be the same, or different. In a desirable embodiment, the substituents are alkoxy e.g., methoxy, ethoxy; or hydroxyl. In a preferred embodiment, Y₂ is a covalent bond.

In another embodiment, B is desirably a substituted aryl group. In an embodiment, A is trisubstituted; two or three of the substituents may be identical. The substituents may be the same, or different. In a desirable embodiment, the substituents are alkoxy, e.g., methoxy, ethoxy; or hydroxyl. In a preferred embodiment, Y₂ is a carbonyl group.

C is desirably H or methyl. In another embodiment, C is a substituted aryl group. In an embodiment, C is trisubstituted; two or three of the substituents may be identical. The substituents may be the same, or different. In a desirable embodiment, the substituents are alkoxy, e.g., methoxy, ethoxy; or hydroxyl. In a preferred embodiment, Y₁ is a carbonyl group.

The compounds of the invention also include prodrug forms of the compound, e.g., phosphate prodrugs.

In another embodiment, the invention provides 2-aryl, 3-aroyl indoles of the following structural formula Ia:

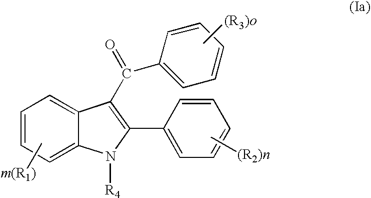

where m, n and o may independently be 0, 1, 2 or 3,

R₁, R₂ and R₃ may be independently selected from OH, nitro, amine, lower alkyl, lower alkoxy, phosphate, or halogen; and

R₄ may be H or lower alkyl, or a substituted aryl group.

In yet another embodiment, the invention provides 2-aroyl, 3-aryl indoles of the following structural formula Ib:

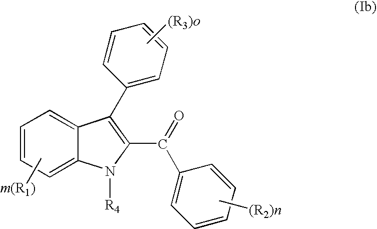

where m, n and o may independently be 0, 1, 2 or 3,

R₁, R₂ and R₃ may be independently selected from OH, nitro, amine, lower alkyl, lower alkoxy, phosphate, or halogen; and

R₄ may be H or lower alkyl, or a substituted aryl group.

In a further embodiment, the invention provides N-aroyl, 2-aryl indoles of the following structural formula Ic:

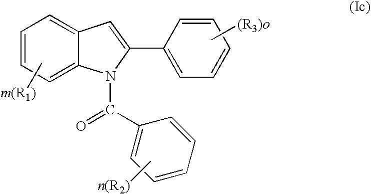

where m, n and o may independently be 0, 1, 2 or 3, and

R₁, R₂ and R₃ may be independently selected from OH, nitro, amine, lower alkyl, lower alkoxy, phosphate, or halogen.

In another embodiment, the invention provides 2,3-diaryl indoles of the following structural formula Id:

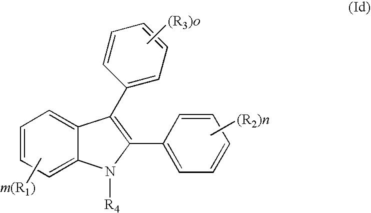

where m, n and o may independently be 0, 1, 2 or 3,

R₁, R₂ and R₃ may be independently selected from OH, nitro, amine, lower alkyl, lower alkoxy, phosphate, or halogen; and

R₄ may be H or lower alkyl, or a substituted aryl group.

In a further embodiment, the invention provides N-aryl, 2-aryl indoles of the following structural formula Ie:

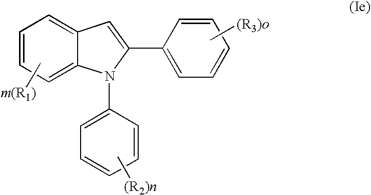

where m, n and o may independently be 0, 1, 2 or 3, and

R₁, R₂ and R₃ may be independently selected from OH, nitro, amine, lower alkyl, lower alkoxy, phosphate, or halogen.

In a second general aspect, the invention contemplates methods of contacting a tubulin-containing system with an effective amount of a compound of Formula I. Methods are also provided for treating a warm-blooded animal afflicted with a neoplastic disease comprising administering an effective amount of compound capable of inhibiting the proliferation of the neoplastic disease. In a preferred embodiment, the antiproliferative effect has the direct result of causing tumor cell cytotoxicity due to inhibition of mitosis.

In a third aspect, the invention broadly contemplates the provision of a method for treating a warm-blooded animal having a vascular proliferative disorder comprising administering an effective amount of a compound of the present invention to achieve targeted vascular toxicity at a locality of proliferating vasculature, wherein the proliferating vasculature is malignant or nonmalignant.

In yet another aspect, the invention broadly contemplates the provision of a method for administering an effective amount of a compound of the present invention to selectively reduce the flow of blood to at least a portion of a neoplastic region, thereby causing substantial necrosis of tissue in the neoplastic region without substantial necrosis of tissue in adjoining regions. In a preferred embodiment, the effect of reduced tumor blood flow is reversible so that normal tumor blood flow is restored following cessation of treatment.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 A) depicts 2-aryl, 3-aroyl-substituted benzo[b]thiophene and benzofuran tubulin binding agents; B) hydroxylated analogs; and C) phosphate prodrugs thereof.

FIG. 2) illustrated the in vivo tumor growth control of the Benzo[b]thiophene prodrug 3-(3′,4′,5′-trimethoxybenzoyl)-2-(4′-methoxyphenyl)-6-methoxybenzo[b]thiophene.

FIG. 3A) illustrates a synthetic route for preparation of phenylindole derivatives involving aryl migration; B) a COSY NMR spectra for the aromatic region of a 2-phenylindole derivative; C) a cyclized isomer without aryl migration (no evidence for its formation).

FIG. 4 illustrates a synthetic route for the preparation of a 2-phenylindole in a one-pot reaction and further derivatization with benzoyl chloride to produce the indole-based analog.

FIG. 5 illustrates a synthetic route for the preparation of a hydroxyl-substituted indole-based ligand and corresponding phosphate prodrug salt.

FIG. 6 illustrates a synthetic route for the preparation of a amino-substituted indole based ligand and corresponding phosphoramidate prodrug salt.

FIG. 7A) depicts exemplary 2-aryl, 3-aroyl-substituted indoles and B) corresponding prodrugs.

FIG. 8A) depicts exemplary 3-aryl, 2-aroyl-substituted indoles and B) corresponding prodrugs.

FIG. 9A) depicts exemplary di- and tri-hydroxylated indole-based ligands; B) depicts exemplary N-substituted indole-based ligands; C) depicts exemplary 2,3-diaryl-substituted indole-based ligands; and D) depicts exemplary 2,3′diaryl ether-substituted indole-based ligands.

FIG. 10 illustrates a synthetic routes for the preparation of dihydroxyl-substituted indole-based ligand and corresponding phosphate prodrug salt.

FIG. 11 illustrates a synthetic route for the preparation of an N-methyl substituted 2-aryl, 3-aroyl-substituted indole prodrug.

FIG. 12 illustrates a synthetic route for the preparation of an N-aroyl, 2-aryl substituted indole prodrug.

FIG. 13 illustrates a synthetic route for the preparation of an exemplary 2,3-diaryl substituted indole and its corresponding diphosphate prodrug.

FIG. 14 illustrates the effect of an exemplary 2-aryl, 3-aroyl-substituted indole phosphate prodrug (Oxi8007) on tumor vasculature over a 24 hour period.

FIG. 15 illustrates the effect of an exemplary 2-aryl, 3-aroyl-substituted indole phosphate prodrug (Oxi8007) on tumor growth.

### DETAILED DESCRIPTION OF THE INVENTION

We have developed a working hypothesis suggesting that the discovery of new antimitotic agents may result from the judicious combination of a molecular template (scaffold) that interacts with estrogen receptor (ER), can be suitably modified with structural features deemed imperative for tubulin binding (i.e. hydroxyl, arylalkoxy groups, certain halogen substitutions, etc.). In particular, the methoxy aryl functionality seems important for increased interaction at the colchicine binding site in certain analogs (Shirai et al., Biomedical Chem. Lett. 1994). Upon formulation of this hypothesis concerning ER molecular templates, our initial design and synthesis efforts centered on benzo [b]thiophene ligands containing structural motifs reminiscent of raloxifene, the selective estrogen receptor modulator (SERM) developed by Eli Lilly and Co. (Jones et al, J. Med Chem. 1984; Grese et al, J. Med Chem., 1997; Palkowitz et al., J. Med Chem., 1997), as well the colchicine and combretastatin tubulin binding agents.

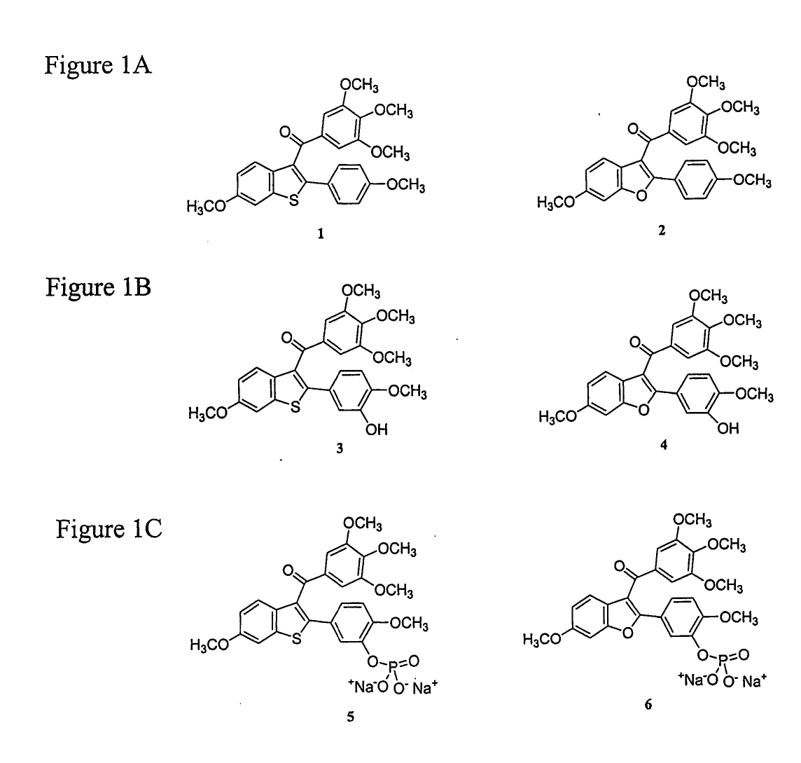

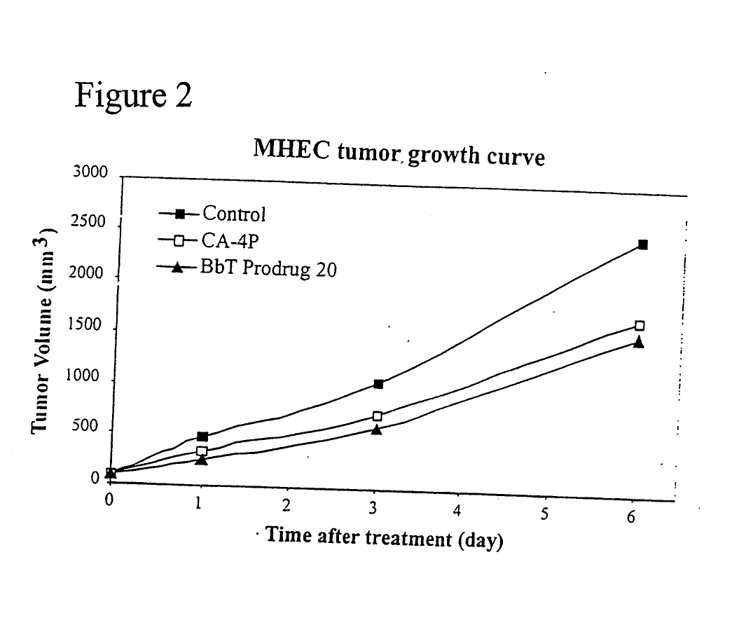

The design premise that molecular skeletons of traditional estrogen receptor (ER) binding compounds can be modified with structural motifs reminiscent of colchicine and combretastatin A4 to produce especially inhibitors of tubulin assembly has been validated by our preparation of very active benzo[b]thiophene and benzo[b]furan antitubulin and antimitotic agents (U.S. Pat. Nos. 5,886,025, 6,162,930, and 6,350,777; Mullica et al., J. Chem. Cryst., 1998; Pinney, et al., Bioorganic and Medicinal Chemistry Letters, 1999; see FIG. 1). The lead compounds in each series demonstrate remarkable biological activity against a variety of human cancer cell lines. For example, 3-(3′,4′,5′- trimethoxybenzoyl)-2-(4′-methoxyphenyl)-6-methoxy benzo[b]thiophene demonstrates potent cytotoxicity and inhibition of tubulin assembly (1, FIG. 1A). In a NCI 60 cell line panel (Boyd et al, Drug Development Research, 1995) this compound produces a mean panel GI₅₀=2.63×10⁻⁷M. In addition, the phenolic derivative of the 3,4-5-trimethoxybenzo[b]thiophene compound (3, FIG. 1B) has pronounced cytotoxicity and demonstrates outstanding inhibition of tubulin assembly while the pro-drug disodium phosphate salt form of this compound (5, FIG. 1C) demonstrates in vitro and in vivo cytotoxicity (U.S. Pat. No. 6,593,374; Pinney et al, ACS Abstract No. 197, 2001). In initial in vivo studies (female Severe Combined Immunodeficient (“SCID”) mice were administered with a single dose (i.p.) of BbT-P (400 mg/kg) one week following inoculation with a MHEC endothelioma tumor (1×10⁻⁶ cells/mouse). These initial studies indicated that the BbT-P compound had some tumor growth control activity (see FIG. 2).

In further support of our hypothesis, recent studies have shown that certain estrogen receptor (ER) binding compounds (e.g., 2-methoxyestradiol) can interact with tubulin and inhibit tubulin assembly as structurally modified estradiol congeners (D'Amato et al., Proc. Natl. Acad Sci,. 1994; Cushman et al., J. Med Chem., 1995; Hamel et al., Biochemistry, 1996; Cushman et al., J. Med. Chem., 1997). Estradiol is perhaps the most important estrogen in humans, and it is intriguing and instructive that the addition of the methoxy aryl motif to this compound makes it interactive with tubulin. It is also noteworthy that 2-methoxyestradiol is a natural mammalian metabolite of estradiol and may play a cell growth regulatory role especially prominent during pregnancy.

The possibility clearly exists that some of the new indole-based ligands described herein, which are structurally related to CA4, may have improved antiproliferative and vascular targeting activity over previously designed compounds. Clearly the ability to selectively disrupt the blood-flow to developing tumor cells is a potential breakthrough in the ever uphill battle against cancer. Certain phenylindoles have been noted for inhibiting tubulin assembly (Gastpar et al., J. Med Chem., 1998) but do not display antivascular properties.

Unlike indole compounds known in the art, the novel indole compounds described in this application are the first to incorporate the 3,4,5-trimethoxyaryl motif reminiscent of colchicine and CA4 arranged in an appropriate molecular conformation such that a pseudo aryl-aryl pi stacking interaction can take place. Such an aryl-aryl interaction of the appropriate centroid-to-centroid distance (approximately 4.7 Angstroms) is believed to be important for enhanced binding affinity to the colchicine site on β-tubulin. It is this binding that ultimately leads to an inhibition of tubulin assembly which manifests itself as a cytotoxic event or antivascular event.

The indole compounds of the present invention demonstrate remarkable cytotoxicity against a variety of human cancer cell lines. The ability of an agent to inhibit tubulin assembly and microtubule formation is an important property of many anticancer agents. Disruption of microtubules that comprise the cytoskeleton and mitotic spindle apparatus can interfere dramatically with the ability of a cell to successfully complete cell division. The compounds of the present invention are highly cytotoxic to actively proliferating cells, inhibiting their mitotic division and often causing their selective apoptosis while leaving normal quiescent cells relatively unaffected.

Nonmalignant Vascular Proliferative Disorders

The invention provides the discovery that the compounds of the invention as well as analogs thereof, are vascular targeting agents (VTAs), and thus are useful for the treatment of malignant vascular proliferative disorders, such as solid tumor cancers, as well as other nonmalignant vascular proliferative disorders, such as retinal neovascularization and restenosis. In one embodiment, the present invention is directed to the administration of a vascular targeting agent, particularly a tubulin binding VTA, for the treatment of malignant or non-malignant vascular proliferative disorders in ocular tissue.

Neovascularization of ocular tissue is a pathogenic condition characterized by vascular proliferation and occurs in a variety of ocular diseases with varying degrees of vision failure. The administration of a VTA for the pharmacological control of the neovascularization associated with non-malignant vascular proliferative disorders such as wet macular degeneration, proliferative diabetic retinopathy or retinopathy of prematurity would potentially benefit patients for which few therapeutic options are available. In another embodiment, the invention provides the administration of a VTA for the pharmacological control of neovascularization associated with malignant vascular proliferative disorders such as ocular tumors.

The blood-retinal barrier (BRB) is composed of specialized nonfenestrated tightly-joined endothelial cells that form a transport barrier for certain substances between the retinal capillaries and the retinal tissue. The nascent vessels of the cornea and retina associated with the retinopathies are aberrant, much like the vessels associated with solid tumors. Tubulin binding agents, inhibitors of tubulin assembly and vascular targeting agents, may be able to attack the aberrant vessels because these vessels do not share architectural similarities with the blood retinal barrier. Tubulin binding agents may halt the progression of the disease much like they do with a tumor-vasculature.

The compounds of the present invention are also contemplated for use in the treatment of vascular disease, particularly atherosclerosis and restenosis. Atherosclerosis is the most common form of vascular disease and leads to insufficient blood supply to critical body organs, resulting in heart attack, stroke, and kidney failure. Additionally, atherosclerosis causes major complications in those suffering from hypertension and diabetes, as well as tobacco smokers. Atherosclerosis is a form of chronic vascular injury in which some of the normal vascular smooth muscle cells (“VSMC”) in the artery wall, which ordinarily control vascular tone regulating blood flow, change their nature and develop “cancer-like” behavior. These VSMC become abnormally proliferative, secreting substances (growth factors, tissue-degradation enzymes and other proteins) which enable them to invade and spread into the inner vessel lining, blocking blood flow and making that vessel abnormally susceptible to being completely blocked by local blood clotting, resulting in the death of the tissue served by that artery.

Restenosis, the recurrence of stenosis or artery stricture after corrective surgery, is an accelerated form of atherosclerosis. Recent evidence has supported a unifying hypothesis of vascular injury in which coronary artery restenosis along with coronary vein graft and cardiac allograft atherosclerosis can be considered to represent a much-accelerated form of the same pathogenic process that results in spontaneous atherosclerosis. Restenosis is due to a complex series of fibroproliferative responses to vascular injury involving potent growth-regulatory molecules, including platelet-derived growth factor (PDGF) and basic fibroblast growth factor (bFGF), also common to the later stages in atherosclerotic lesions, resulting in vascular smooth muscle cell proliferation, migration and neointimal accumulation.

Restenosis occurs after coronary artery bypass surgery (CAB), endarterectomy, and heart transplantation, and particularly after heart balloon angioplasty, atherectomy, laser ablation or endovascular stenting (in each of which one-third of patients redevelop artery-blockage (restenosis) by 6 months), and is responsible for recurrence of symptoms (or death), often requiring repeat revascularization surgery. Despite over a decade of research and significant improvements in the primary success rate of the various medical and surgical treatments of atherosclerotic disease, including angioplasty, bypass grafting and endarterectomy, secondary failure due to late restenosis continues to occur in 30-50% of patients.

The most effective way to prevent this disease is at the cellular level, as opposed to repeated revascularization surgery which can carry a significant risk of complications or death, consumes time and money, and is inconvenient to the patient.

Definitions

As used herein, the following terms in quotations shall have the indicated meanings, whether in plural or singular form:

“Amino acid acyl group” in the amino acid acylamino group includes an acyl group derived from the amino acid. The amino acids may be enumerated by α-amino acids, β-amino acids and γ-amino acids. Examples of preferred amino acids include glycine, alanine, leucine, serine, lysine, glutamic acid, aspartic acid, threonine, valine, isoleucine, ornithine, glutamine, asparagines, tyrosine, phenylalanine, cysteine, methionine, arginine, β-alanine, tryptophan, proline, histidine, etc. The preferred amino acid is serine and the preferred amino acid acyl group is a serinamide.

“Amine” includes a free amine NH₂ or a lower alkylamino.

“Animal” includes any warm-blooded mammal, preferably a human.

“Alkyl” includes a group containing from 1 to 8 carbon atoms and may be straight chained or branched. An alkyl group is an optionally substituted straight, branched or cyclic saturated hydrocarbon group. When substituted, alkyl groups may be substituted with up to four substituent groups, R as defined, at any available point of attachment. When the alkyl group is said to be substituted with an alkyl group, this is used interchangeably with “branched alkyl group”. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Exemplary substituents may include but are not limited to one or more of the following groups: halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). Alkyl groups as defined may also comprise one or more carbon to carbon double bonds or one or more carbon to carbon triple bonds.

“Aryl” includes groups with aromaticity, including 5- and 6-membered single-ring aromatic groups that may include from zero to four heteroatoms, as well as multicyclic systems with at least one aromatic ring. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. The aromatic ring can be substituted at one or more ring positions with such substituents as described above, as for example, halogen, hydroxyl, alkoxy, etc. The preferred aryl group of the present invention is an optionally-substituted benzene ring of the following structure

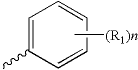

where R₁ is OH, nitro, amine, lower alkyl, lower alkoxy, carboxyl, or halogen and n is 0, 1, 2, 3, 4, or 5.

“Aroyl” includes the —(C═O)-aryl groups, wherein aryl is defined as hereinabove. The aryl group is bonded to the core compound through a carbonyl bridge.

“Cycloalkyl” is a species of alkyl containing from 3 to 15 carbon atoms, without alternating or resonating double bonds between carbon atoms. It may contain from 1 to 4 rings. Exemplary unsubstituted such groups include cyclopropyl, cyclobutyl, cyclopentyl, cyclohexyl, adamantyl, etc. Exemplary substituents include one or more of the following groups: halogen, alkyl, alkoxy, alkyl hydroxy, amino, nitro, cyano, thiol and/or alkylthio.

“Halogen” or “Halo” includes chlorine, bromine, fluorine or iodine.

“Lower alkoxy” includes —O—alkyl groups, wherein alkyl is as defined hereinabove. The alkoxy group is bonded to the core compound through the oxygen bridge. The alkoxy group may be straight chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy.

“Lower alkylamino” includes a group wherein one or two alkyl groups is bonded to an amino nitrogen, i.e., NH(alkyl). The nitrogen is the bridge connecting the alkyl group to the core compound. Examples include NHMe, NHEt, NHPr, and the like.

“Prodrug” includes a precursor form of the drug which is metabolically converted in vivo to produce the active drug. Preferred prodrugs of the present invention include phosphate, phosphoramidate, or amino acid acyl groups as defined herein. The phosphate ester salt moiety may also include (—OP(O)(O-alkyl)₂ or (—OP(O)(O—NH₄⁺)₂).

“Phenolic moiety” means herein a hydroxy group when it refers to an R group on an aryl ring.

“Phosphate”, “Phosphate moiety”, or “Phosphate prodrug salt” includes phosphate ester salt moiety (—OP(O)(O⁻M⁺)₂), a phosphate triester moiety (—OP(O)(OR)₂) or a phosphate diester moiety (—OP(O)(OR)(O⁻M⁺), where M is a salt and R is chosen to be any appropriate alkyl or branched alkyl substituent (the two R groups may be the same alkyl group or may be mixed), or benzyl, or aryl groups. The salt M is advantageously Na, K and Li, but the invention is not limited in this respect.

“Phosphoramidate” includes a phosphoamidate ester salt moiety (—NP(O)(O⁻M⁺)₂), a phosphoramidate diester moiety (—NP(O)(OR)₂), or a phosphamidate disalt moiety (—NP(O)(OR)(O⁻M⁺), where M is a salt and R is chosen to be any appropriate alkyl or branched alkyl substituent (the two R groups may be the same alkyl group or may be mixed), or benzyl, or aryl groups. The salt M is advantageously Na, K and Li, but the invention is not limited in this respect.

“Salt” is a pharmaceutically acceptable salt and can include acid addition salts such as the hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na, K, Li, alkaline earth metal cations such as Mg⁺² or Ca⁺², or organic amine salts such as those disclosed in PCT International Application Nos. WO02/22626 or WO00/48606.

“Tubulin Binding Agent” shall refer to a ligand of tubulin or a compound capable of binding to either αβ-tubulin heterodimers or microtubules and interfering with the assembly or disassembly of microtubules.

“Tumors”, “Cancers”, or “Neoplastic Disease” shall be used interchangeably and include (but are not limited to) the following:
1) carcinomas, including that of the bladder, breast, colon, rectum, kidney, liver, lung (including small cell lung cancer), pharynx, esophagus, gall bladder, urinary tract, ovaries, cervix, uterus, pancreas, stomach, endocrine glands (including thyroid, adrenal, and pituitary), prostate, testicles and skin, including squamous cell carcinoma;
2) hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkett's lymphoma;
3) hematopoietic tumors of myeloid lineage, including acute and chronic myelogenous leukemias, myelodysplastic syndrome and promyelocytic leukemia;
4) tumors of mesenchymal origin, including fibrosarcoma and rhabdomyosarcoma;
5) tumors of the central and peripheral nervous system and meninges, including astrocytoma, neuroblastoma, glioma, schwannomas, retinoblastomas, neuroma, glioma, gliobalstoma; and
6) other tumors, including melanoma, seminoma, teratocarcinoma, osteosarcoma, xenoderoma pigmentosum, keratoctanthoma, thyroid follicular cancer, anaplastic thyroid cancer and Kaposi's sarcoma.

“Vascular toxicity” includes the selective destruction, damage, or occlusion, whether reversible or irreversible, partial or complete, of proliferating vasculature.

“Malignant proliferating vasculature” includes the endothelium, artery, blood vessel, or neovasculature formed by a malignant disease state, such as a tumor.

“Nonmalignant proliferating vasculature” includes the endothelium, artery, blood vessel, or neovasculature formed by undesirable or pathological angiogenesis and that is associated with disease states other than a malignant disease state, including without limitation, ocular diseases such as wet or age-related macular degeneration, myopic macular degeneration, diabetic retinopathy, retinopathy of prematurity, diabetic molecular edema, uveitis, neovascular glaucoma, rubeosis, retrolental fibroplasias, angioid streaks, ocular histoplasmosis, and corneal neovascularization, or other nonocular disease states such as atherosclerosis, endometriosis, psoriasis, rheumatoid arthritis, Osler-Webber Syndrome, wound granulation, atheroma, restenosis, Kaposi's sarcoma, haemangioma, restenosis, and in general, inflammatory diseases characterized by vascular proliferation.

“Antiproliferative” or “antimitotic” refer to the ability of the compounds of the present invention to directly inhibit the proliferation of tumor cells and impart direct cytotoxicity towards tumor cells.

“Treating” (or “treat”) as used herein includes its generally accepted meaning which encompasses prohibiting, preventing, restraining, and slowing, stopping, or reversing the progression or severity of a resultant symptom. As such, the methods of this invention encompass both therapeutic and prophylactic administration.

“Effective amount” includes the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of, involvement, or the severity of the disease; the response of the individual patient; the particular compound administered; the mode of administration; the bioavailability characteristics of the preparation administered; the dose regimen selected; the use of concomitant medication; and other relevant circumstances.

Dosage and Administration of Compounds

A typical daily dose will contain from about 0.1 mg/kg to about 1000 mg/kg of the active compound of this invention. Preferably, daily doses will be about 10 mg/kg to about 100 mg/kg, and most preferably about 10 mg.

In effecting treatment of a patient afflicted with a condition, disease or disorder described herein, a compound of the present invention can be administered systemically in any form or mode which makes the compound bioavailable in effective amounts. Systemic administration may be accomplished by administration of a compound of the present invention into the bloodstream at a site which is separated by a measurable distance from the diseased or affected organ or tissue. For example, compounds of the present invention can be administered orally, parenterally, subcutaneously, intramuscularly, intravenously, transdermally, intranasally, rectally, buccally, and the like. Oral or intravenous administration is generally preferred for treating neoplastic disease or cancer. Alternatively, the compound may be administered non-systemically by local administration of the compound of the present invention directly at the diseased or affected organ or tissue. Treatment of ocular diseases characterized by the presence of non-malignant proliferative vasculature or neovascularization, can be achieved using non-systemic administration methods such as intravitreal injection, sub-Tenon's injection, ophthalmic drops, iontophoresis, topical formulation, and implants and/or inserts. One skilled in the art of preparing formulations can readily select the proper form and mode of administration depending upon the particular characteristics of the compound selected, the disease state to be treated, the stage of the disease, and other relevant circumstances.

It will be understood by the skilled reader that all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

According to another aspect, the present invention provides a pharmaceutical composition, which comprises a compound of the present invention or a pharmaceutically acceptable salt thereof as defined hereinabove and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well know in the art.

The compositions are preferably formulated in a unit dosage form, each dosage containing from about 1 mg to about 500 mg, more preferably about 5 mg to about 300 mg (for example 25 mg) of the active ingredient. The term “unit dosage form” includes a physically discrete unit suitable as unitary dosages for human subjects and other mammals, each unit containing a predetermined quantity of active material calculated to produce the desired therapeutic effect, in association with a suitable pharmaceutical carrier, diluent, or excipient. The following formulation examples are illustrative only and are not intended to limit the scope of the invention in any way.

The compositions of the present invention may be formulated in a conventional manner using one or more pharmaceutically acceptable carriers. Thus, the active compounds of the invention may be formulated for oral, buccal, transdermal (e.g., patch), intranasal, parenteral (e.g., intravenous, intramuscular or subcutaneous) or rectal administration or in a form suitable for administration by inhalation or insufflation.

Alternatively, compounds of the present invention can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are well known in the art (see for example, Prescott Ed., Methods in Cell Biology, Volume XIV, Academic Press, New York, N.Y., 1976, p 33).

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose of calcium phosphate); lubricants (e.g., magnesium stearate, talc or silica); disintegrants (e.g., potato starch or sodium starch glycollate); or wetting agents (e.g., sodium lauryl sulphate). The tablets may be coated by methods well known in the art. Liquid preparations for oral administration may take the form, of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in a powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension form a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

Tablets or capsules of the compounds may be administered singly or two or more at a time as appropriate. It is also possible to administer the compounds in sustained release formulations.

The physician will determine the actual dosage which will be most suitable for an individual patient and it will vary with the age, weight and response of the particular patient. The above dosages are exemplary of the average case. There can of course, be individual instances where higher or lower dosage ranges are merited, and such are within the scope of this invention.

The compounds of the present invention can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required.

“Administering” means any of the standard methods of administering a compound to a subject, known to those skilled in the art. Examples include, but are not limited to, intravenous, intramuscular or intraperitoneal administration.

For the purposes of this invention “pharmaceutically acceptable carriers” means any of the standard pharmaceutical carriers. Examples of suitable carriers are well known in the art and may include, but are not limited to, any of the standard pharmaceutical carriers such as a phosphate buffered saline solutions, including phosphate buffered saline solutions containing POLYSORB 80, water, emulsions such as oil/water emulsion, and various type of wetting agents. Other carriers may also include sterile solutions, tablets, coated tablets, and capsules.

Typically such carriers contain excipients such as starch, milk, sugar, certain types of clay gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

Methods of determining an “effective amount” are well known to those skilled in the art and depend upon factors including, but not limited to, the size of the patient and the carrier used.

Combination Therapies for Solid Tumor Cancers

The compounds of the present invention may also be useful for the treatment of solid tumor described above when used either alone or in combination with radiotherapy and/or other chemotherapeutic treatments conventionally administered to patients for treating solid tumor cancers. For example, compounds of the present invention may be, administered with chemotherapeutic agents selected from one of the following functional classes:
1. Alkylating agents: compounds that donate an alkyl group to nucleotides. Alkylated DNA is unable to replicate itself and cell proliferation is stopped. Exemplary alkylating agents include Melphalan, Chlorambucil, cyclophosphamide, ifosfamide, busulfan, dacarbaine, methotrexate, 5-FU, cytosine arabinsoide, and 6-thioguanine.
2. Antiangiogeneic agents: compounds that inhibit the formation of tumor vasculature. Exemplary anti-angiogenic agents include TNP-470 and Avastin™.
3. Antitumor Antibiotics: compounds having antimicrobial and cytotoxic activity. Such compounds also may interfere with DNA by chemically inhibiting enzymes and mitosis or altering cellular membranes. Exemplary antitumor antibiotics include Actinomycin-D, bleomycin, mitomycin-C, Dactinomycin, Daunorubicin, and Doxorubicin.
4. Topoisomerase Inhibitors: agents which interfere with topoisomerase activity thereby inhibiting DNA replication. Such agents include CPT-11 and Topotecan.
5. Hormonal Therapy: includes, but is not limited to anti-estrogens. An exemplary antiestrogen is Tamoxifen.
6. Antimicrotubule compounds: Vincristine, paclitaxel, taxotere, etoposide, vinblastine, etc.

### EXAMPLES

The invention is further described by reference to the following examples and preparations which describe the manner and process of making and using the invention and are illustrative rather than limiting. It will be apparent to those skilled in the art that many modifications, both to the materials and methods, may be practiced without departing from the purpose and interest of the invention.

Materials and Methods

Chemicals were obtained commercially from the Aldrich Chemical Company, Fisher Scientific, and ACROS Chemicals and used directly as purchased. Solvents such as ethylacetate, acetone, and diethyl ether, were used as purchased, and others were purified by standard procedures as necessary. Methylene chloride (CH₂Cl₂) was dried over calcium hydride or sodium hydride and distilled immediately prior to use. Hexanes used for the flash chromatography was distilled from calcium hydride.

Reactions were followed by thin layer chromatography (TLC), and/or gas chromatography mass spectrometry (GC-MS). Products were purified using flash column chromatography with silica gel (260-400 mesh), preparatory thin layer chromatography or recrystallization. Silica gel plates for thin layer chromatography (TLC), preparatory chromatography, and silica gel (200-400 mesh) for column chromatography were obtained from Merck EM Science.

Proton (¹H), (¹³C) and (³¹P) spectra were recorded with a Bruker 300 spectrometer operating at 300 MHz for proton, 75 MHz for carbon and 120 MHz for phosphorous, unless otherwise stated. All NMR spectra were recorded in the indicated solvent at room temperature. Infrared spectrum was obtained as Nujol suspension on NaCl plates and was recorded on Genesis II Fourier-transform infrared spectrometer. Chemical shifts are expressed in ppm (δ), peaks are listed as singlet (s), doublet (d), triplet (t) or multiplet (m), with coupling constant (J) expressed in Hz.

Elemental analyses were obtained from Atlantic Microlab Inc., Norcross, Ga. Melting points were determined using a Thomas-Hoover melting point apparatus and are uncorrected.

### Example 1

### Synthesis of 2-Aryl, 3-Aroyl Substituted Indole Tubulin Binding Agents

A) Synthesis of Phenylindoles

Method I (2 Steps):

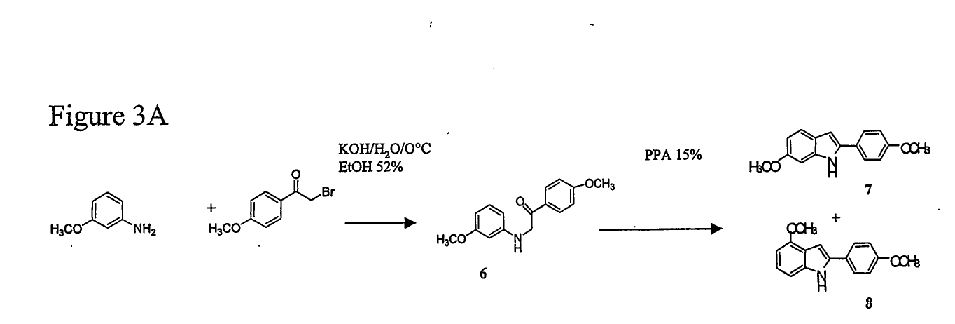

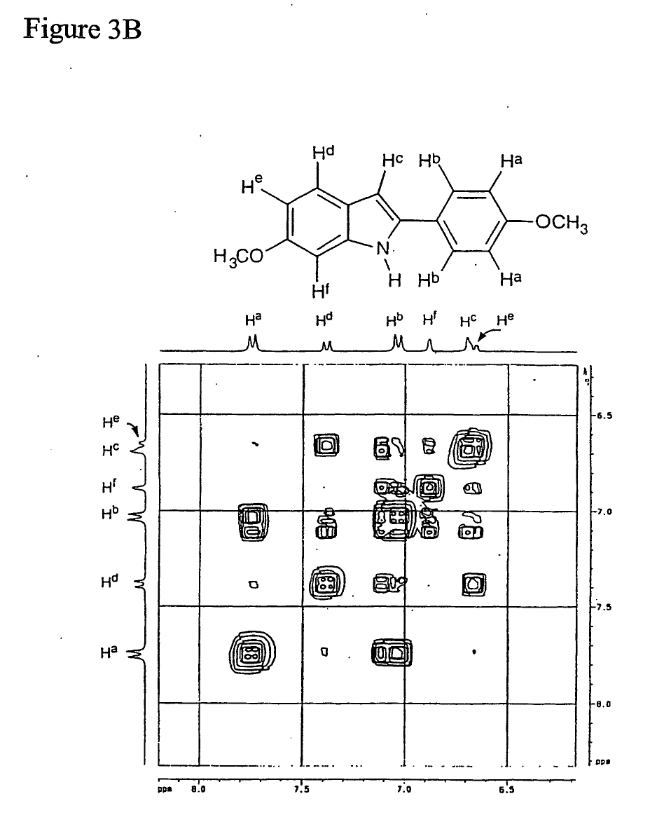

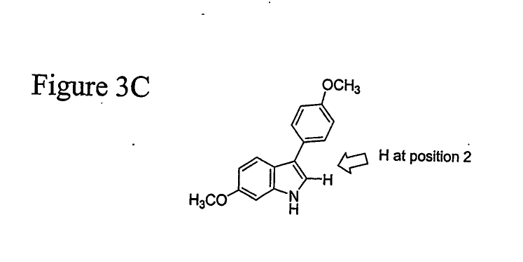

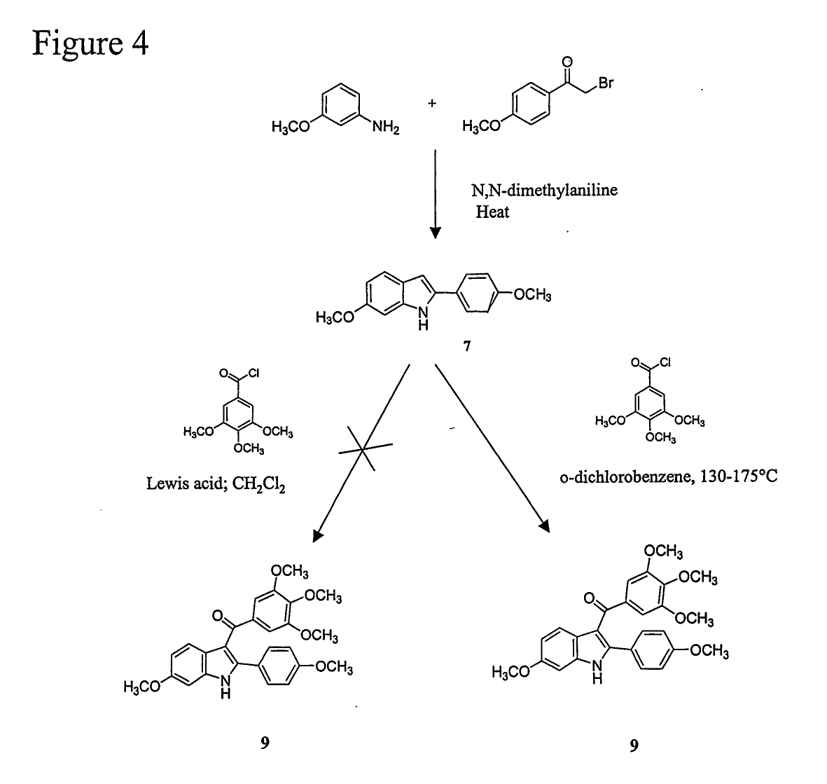

Indole-based ligand 9 was synthesized as shown in FIGS. 3 and 4.

Secondary amine 6 was prepared by treatment of m-anisidine and 2-bromo-4-methoxyacetophenone under basic conditions (ethanolic potassium hydroxide) at 0° C. To a well-stirred solution of KOH (0.926 g, 16.5 mmol) in EtOH (18 ml) and H₂O (9 ml) at R.T. was added m-anisidine (2.192 g, 17.80 mmol) by syringe. The solution was then stirred at 0° C. After 10 minutes, the solution of 2-bromo-4-methoxyacetophenone (4.09 g, 17.80 mmol) was added dropwise with an addition funnel over a 40 minute period. After 24 h, 0° C. to R.T., water was added. The product was isolated by extraction (¹H HCl, NaHCO₃, brine, MgSO₄). The product was purified by recrystallization (50:50 EtOAc:hexanes) to afford secondary amine 6 (2.46 g, 9.07 mmol, 52%) as a yellow solid:

H NMR (CDCl₃): δ7.98 (2H, d, J=8.9 Hz), 7.12 (1H, t, J 8.1 Hz), 6.97 (2H, d, J 8.9 Hz), 6.30 (3H, m), 4.54 (2H, s), 3.88 (3H, s), 3.79 (3H, s).

Secondary amine 6 (4.0 g, 14.74 mmol) was added in 6 portions over a 30 minute period to a round-bottom flask charged with polyphosphoric acid (PPA). After 2 hours at 80° C. to 90° C., water was added. The product was isolated by extraction (EtOAc, NaHCO₃, brine, MgSO₄) and shown to consist of two regioisomers, 6-methoxyphenylindole (7) and 4-methoxyphenylindole (8). These isomers have poor solubility in EtOAc, CH₂Cl₂ and EtOH. Phenylindole 7 was purified (from indole 8) by trituration in acetone. Purification by recrystallization (acetone) afforded the indole 7 (0.544 g, 2.15 mmol, 15%) as a pale yellow solid.

H NMR (CDCl₃): δ 11.24 (1H, br, s), 7.72 (2H, d, J 8.82 Hz), 7.36 (1H, d, J=8.57 Hz), 7.00 (2H, d, J=8.84 Hz), 6.85 (1H, d, J=2.07 Hz), 6.66 (1H, d, J=1.66 Hz), 6.63 (1H, dd, J 8.59, 2.28 Hz), 3.78 (3H, s), 3.77 (3H, s). ¹³C NMR (CDCl₃): δ 158.15, 155.22, 137.44, 136.33, 125.60, 124.93, 122.82, 120.04, 114.07, 109.00, 96.97, 94.01, 54.93, 54.88.

The structure of the 7 isomer was confirmed by NMR analysis. COSY NMR was taken in order to study, in detail, the coupling relationship between the protons. The enlarged COSY spectrum for the aromatic region of phenylindole 7 is shown in FIG. 3B. This COSY NMR spectrum showed a strong coupling between H^{a} and H^{b} and appears as a doublet. H^{c} is coupled by the proton attached to the nitrogen into a small doublet. H^{d} is coupled only by H^{e} into a corresponding doublet, while H^{e} is coupled both by an ortho coupling (H^{d}) and by a meta coupling (H^{f}) into a doublet of doublet pattern. H^{f} is coupled by H^{e} into a doublet. Further evidence of the formation of phenylindole 7 is the chemical shift of the proton H^{c} on the ring which contains nitrogen. Through computer modeling (ChemDraw Ultra 4.5), the theoretical chemical shift value of 6.4 ppm was predicted for proton H^{c} (at the 3 position) and matches the peak shown in the actual NMR spectrum at 6.6 ppm. For the case where the proton is at the 2 position (FIG. 3C), the chemical shift was predicted to be 7.03 ppm, which does not match any peak in the spectrum that was obtained. Based collectively on these studies, the formation of phenylindole 7 was confirmed, and the migration of the methoxyphenyl system was evidenced. The other isomer (phenylindole 8) is soluble in acetone and was much more difficult to obtain in pure form.

Method II (1 Step):

The alternative one-pot methodology of Von Angerer and co-workers (Von Angerer et al, J. Med Chem. 1984) was also applied to the preparation of the desired 2-phenylindole (FIG. 4).

To a boiling mixture of m-anisidine (1.56 ml, 20.0 mmol) and N,N-dimethylaniline (3.5 ml) was added 2-bromo-4-methoxyacetophenone (1.37 g in EtOAc, 6.00 mmol) slowly by syringe. After addition, the mixture was kept at 170° C. for 1 hour. The reaction mixture was cooled to room temperature and a dark-colored solid was formed. EtOAc was added along with HCl (2 N). The aqueous layer was extracted with EtOAc several times. The combined organic layers were washed with brine, and dried over MgSO₄. Solvent was removed under the reduced pressure to afford a dark brown-colored solid. Purification by recrystallization in EtOH afforded indole 7 as a white crystalline material.

H NMR(CDCl₃): δ 11.24 (1H,br,s), 7.72(2H,d, J 8.82 Hz), 7.36 (1H,d, J 8.57 Hz),7.00 (2H,d, J=8.84 Hz), 6.85 (1H, d, J=2.07 Hz), 6.66 (1H, d, J=1.66 Hz), 6.63 (1H, dd, J 8.59, 2.28 Hz),3.78 (3H, s), 3.77 (3H, s). ¹³C N (CDCl₃): δ 158.15, 155.22, 137.44, 136.33, 125.60, 124.93, 122.82, 120.04, 114.07, 109.00, 96.97, 94.01, 54.93, 54.88; Melting Point: 208-229.5° C.; HRMS (E1) M+ calcd for CH₁₆NO₂ 253.3035, found 253.1060.

B) Synthesis of 2-aryl, 3-aroyl indoles

In order to synthesize the 2 aryl, 3-aroyl indole-based analog 9, Friedel-Crafts acylation was carried out (FIG. 4). Treatment of phenylindole 9 with 3,4,5-trimethoxybenzoyl chloride in the presence of the Lewis-Acid AlCl₃ failed to yield the desired compound. Attempts to modify the reaction conditions by increasing the reaction temperature or employing other Lewis Acids (ex. TiCl₄) proved futile as well, as starting material was recovered in all cases. One possible explanation for this result is the fact that the nitrogen atom (containing a lone pair of electrons and an acidic proton) may disrupt the acylation process. According to this analysis, a Grignard reagent (ethylmagnesium bromide) was used to protect this nitrogen prior to the Friedel-Crafts acylation step. Still, only starting material was obtained following the reaction. Therefore, a new synthetic approach was brought into this study.

In 1977, Inion and co-workers reported the synthesis of a variety of aminoalkoxy-4-benzoyl-3-indoles (Inion et al., Eur. J. Med. Chem. 1977). The benzoate indole product was prepared by treatment of the corresponding indole with an appropriate benzoyl chloride and heating (130-150° C.). HCl was generated under these conditions. Therefore, a similar synthetic approach was used in the synthesis of the desired 2-aryl, 3-aroyl substituted indole ligand 9 (FIG. 4). Since both the precursor, phenylindole 7, and trimethoxybenzoyl chloride are solid, a solvent with a high boiling point was needed. 1,2-dichlorobenzene was chosen in this case since it has a boiling point of 180° C.

To a well stirred solution of phenylindole 7 (0.502 g, 1.98 mmol) in o-dichlorobenzene (10 ml) was added trimethoxybenzoylchloride (0.692 g, 3.00 mmol). The reaction mixture was heated to reflux for 12 hours. Solvent was removed by distillation under reduced pressure. After cooling down to room temperature, a dark solid formed which was dissolved in chloroform and purified by silica gel column chromatography with chloroform as the eluent. The collected mixture was again purified by column chromatography (50:50 hexanes:EtOAc) affording trimethoxybenzoyl indole 9 (0.744 g, 1.66 mmol, 84%) as a yellow oily gel. Pale yellow-green crystals were obtained by recrystallization from a mixture of ethanol and hexanes.

1H NMR (CDCl₃): δ 8.63 (1H, br, s), 7.88 (1H, d, J=9.39 Hz), 7.24 (2H, d, J=8.78 Hz), 6.95(2H, s 6.90 (2H, m), 6.71 (2H, d, J=8.79 Hz), 3.86 (3H, s), 3.80 (3H, s), 3.73 (3H, s), 3.68 (6H, s); ¹³C NMR (CDCl₃): δ 192.23, 159.73, 157.06, 152.42, 142.85, 141.01, 136.41, 134.65, 130.16, 124.28, 122.94, 122.17, 113.67, 112.46, 111.52, 107.24, 94.54, 60.78, 55.92, 55.54, 55.14; Melting Point: 153-155° C.; Anal. Calcd for C₂₆H₂₅NO₆: C, 69.79; H, 5.63; H, 3.13. Found: C, 69.61; H, 5.63; N, 3.01.

### Example 2

### Synthesis of Hydroxylated 2-Aryl, 3-Aroyl Substituted Indoles and Corresponding Phosphate Prodrugs

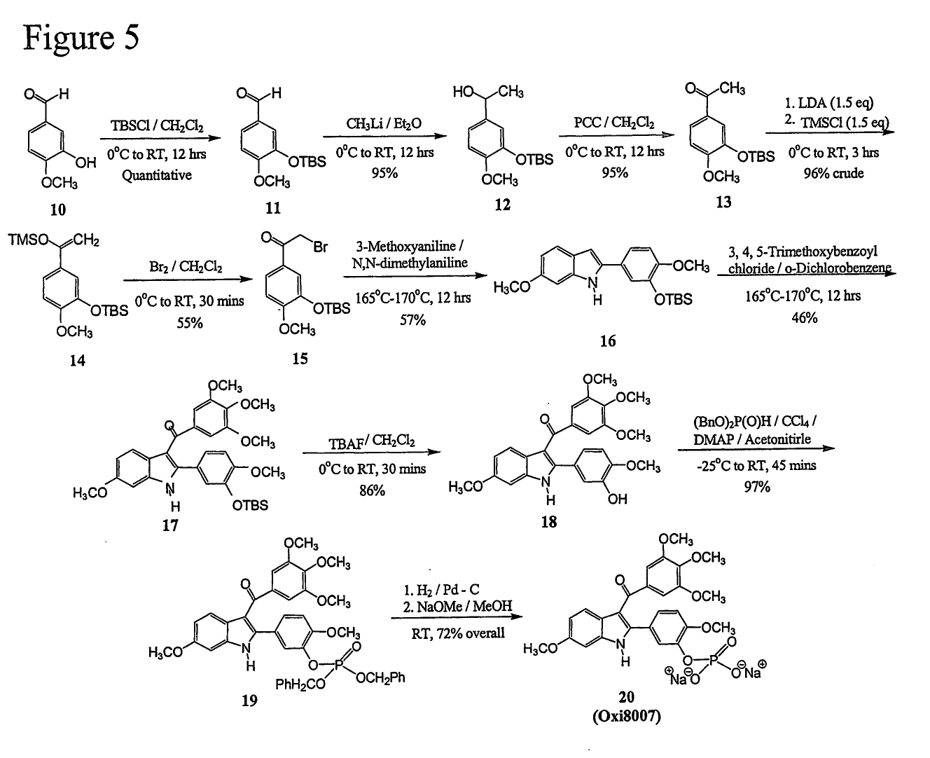

Based on promising results obtained with benzo[b]thiophene and benzofuran analogs, the preparation of hydroxylated indoles and corresponding phosphate salts was conducted as illustrated in FIG. 5.

A) Synthesis of Hydroxylated Bromoacetophenone

The synthesis of the target indole phosphate molecule involved the synthesis of a substituted bromoacetophenone 15 as a key intermediate, using commercially available isovanillin 10 (FIG. 4). After the protection of the phenol as silyl ether 11, addition of methyllithium gave the secondary alcohol 12 and this on oxidation with PCC gave the expected acetophenone derivative 13. Reaction of 13 with LDA, generated in situ, followed by addition of bromine resulted in the desired bromoacetophenone 15.

i) 3-tert-Butyldimethylsilyloxy-4-methoxybenzaldehyde (11)

To a well-stirred solution of 3-hydroxy-4-methoxybenzaldehyde 10 (15.26 g, 100.39 mmol) in CH₂Cl₂ (200 mL) under argon, was added Et₃N (15.4 mL, 110.43 mmol) followed by N,N-dimethylaminopyridine (1.22 g, 10.04 mmol) and tert-butyldimethylsilyl chloride (16.62 g, 110.43 mmol) at 0° C. The reaction mixture was allowed to warm to room temperature and stirred for 12 hours. Then, the solids were separated by filtration, water (100 mL) was added and the phases were separated. The organic phase was dried over Na₂SO₄ and concentrated in vacuo to afford compound 11 as yellowish oil (26.81 g, 100.79 mmol) in quantitative yields. The isolated product was found to be very pure by NMR analysis and hence was carried to the next step without further purification.

¹H-NMR (300 MHz, CDCl₃): δ 9.84 (s, 1H), 7.49 (dd, J=2.1, 8.2 Hz, 1H), 7.48 (d, J=2.1 Hz, 1H), 6.98 (d, J=8.2 Hz, 1H), 4.04 (s, 3H), 1.03 (s, 9H), 0.20 (s, 6H). ¹³C-NMR (75 MHz, CDCl₃): 190.8, 156.5, 145.4, 130.0, 126.3, 119.8, 111.0, 55.4, 25.5, 18.3, −4.8.

ii) 3-tert-Butyldimethylsilyloxy-1-(1′-hydroxyethyl)-4-methoxybenzene (12)

To a solution of 11 (26.61 g, 100.04 mmol) in anhydrous diethyl ether (250 mL) was added MeLi (150.0 mL, 1.4 M in Et₂O, 210.08 mmol), dropwise at 0° C. under argon. The reaction mixture was stirred for 2 hours allowing it to reach the room temperature. Then, the flask was cooled to 0° C., the reaction was slowly quenched with water and the phases were separated. The organic phase was dried over Na₂SO₄ and the removal of solvent afforded alcohol 12 (26.84 g, 95.17 mmol, 95%) as a pale yellow oil, which was carried to the next step without further purification.

¹H-NMR (300 MHz, CDCl₃): δ 6.85 (m, 2H), 6.78 (d, J=9.5 Hz, 1H), 4.75 (q, J=5.3 Hz, 1H), 3.76 (s, 3H), 1.42 (d, J=5.3, 3H), 0.97 (s, 9H), 0.13 (s, 6H).

iii) 3-tert-Butyldimethylsilyloxy-4-methoxyacetophenone (13)

Celite (50.0 g) was added to a cold solution of 12 (114.2 g, 404.96 mmol) in dry CH₂Cl₂ (800 mL) followed by pyridiniumchlorochromate (96.02 g, 445.46 mmol) in portions. The reaction mixture was warmed to room temperature and then stirred for 12 hours. Then, it was filtered through a short pad of celite/silica gel (50/50) rinsing well with CH₂Cl₂. The residue was concentrated onto silica gel under reduced pressure and subjected to flash column chromatography (silica gel, hexanes/EtOAc: 80/20) to afford 107.9 g (385.3 mmol, 95%) of the TBS-protected ketone 13 as pale yellow crystalline solid.

¹H-NMR (300 MHz, CDCl₃): δ 7.49 (dd, J=2.1, 8.4 Hz, 1H), 7.38 (d, J=2.1 Hz, 1H), 6.77 (d, J=8.4 Hz, 1H), 3.77 (s, 3H), 2.43 (s, 3H), 0.91 (s, 9H), δ 0.08 (s, 6H). ¹³C-NMR (75 MHz, CDCl₃): 196.7, 155.3, 144.8, 130.6, 123.5, 120.5, 110.8, 55.5, 26.3, 25.7, 18.4, 4.6.

iv) 1-(3-tert-Butyldinethylsilyloxy-4-methoxyphenyl)-1-trimethylslaylethene (14)

In a dry round bottom flask under argon was placed dry ether (100 mL) and diisopropylamine (15.01 mL, 107.14 mmol) and the solution was cooled to 0° C. To this cold solution, butyllithium (41.18 mL of a 2.6M solution, 107.1 mmol) was added dropwise. Meanwhile, the TBS-protected acetophenone 13 (20.00 g, 71.43 mmol) was weighed into a 250 mL flask, put under argon, dissolved in dry ether (100 mL) and this solution was added dropwise, with good stirring to the cold LDA solution. After 5 minutes, trimethylsilyl chloride (13.6 mL, 107.1 mmol) was added dropwise and the solution was allowed to stir for 3 hours. The reaction was then quenched with 10% NaHCO₃ (200 mL) followed by extraction with hexanes (300 mL), and dried over K₂CO₃. Filtration and concentration by rotary evaporation gave the crude TMS enol ether 14 (24.1 g, 68.3 mmol, 96%) as yellow oil. The isolated product was found to be very pure by NMR analysis and hence was carried to the next step without further purification.

¹H-NMR (300 MHz, CDCl₃): δ 7.17 (dd, J=2.2, 8.4 Hz, 1H), 7.11 (d, J=2.2 Hz, 1H), 6.78 (d, J=8.4 Hz, 1H), 4.77 (d, J=1.6 Hz, 1H), 4.33 (d, J=1.6 Hz, 1H), 3.8 (s, 3H), 1.02 (s, 9H), 0.26 (s, 9H), 0.16 (s, 6H). (75 MHz, CDCl₃): 155.2, 151.1, 144.4, 130.5, 118.8, 118.1, 111.3, 89.5, 55.4, 25.7, 18.4, 0.04, −4.7.

v) 3′-tert-Butyldimethlysilyloxy-4′-methoxy-2-bromoacetophenone (15)

To a solution of the crude 14 (24.1 g, 68.3 mmol) in dry CH₂Cl₂ (100 mL) was added 1.00 g of powdered anhydrous K₂CO₃ and the mixture was cooled to 0° C. under argon. Then bromine (3.1 mL, 61.5 mmol) was added slowly, dropwise. After stirring for few minutes, the reaction was worked up by adding sodium thiosulfate solution. The phases were separated and the aqueous phase was extracted with CH₂Cl₂ (3×50 mL). The combined organic solutions were dried over Na₂SO₄ and the deep red oil was concentrated under reduced pressure. It was then dissolved in 80 mL of hexanes and placed in the refrigerator overnight. This resulted in 13.5 g (37.6 mmol, 55%) of the desired bromoacetophenone derivative 15 as red crystals.

¹H-NMR (300 MHz, CDCl₃): δ 7.62 (dd, J=2.2, 8.5 Hz, 1H), 7.49 (d, J=2.2 Hz, 1H), 6.89 (d, J=8.5 Hz, 1H), 4.38 (s, 2H), 3.87 (s, 3H), 1.00(s, 9H), 0.17 (s, 6H). ¹³C-NMR (75 MHz, CDCl₃): 189.9, 156.1, 145.1, 127.1, 124.2, 121.1, 111.0, 55.5, 30.7, 25.6, 18.4, −4.6.

B) Synthesis of Hydroxylated Phenylindole

Condensation of the bromoacetophenone 15 with 3-methoxyaniline gave the 2-substituted indole derivative 16. Reaction of 16 with 3,4,5-trimethoxybenzoyl chloride yielded the 3- trimethoxybenzoyl substituted indole derivative 17 in a Friedel-Craft's benzoylation fashion. Desilylation of 17 with TBAF gave the desired hydroxylated indole ligand 18. Reaction of 18 with in situ-generated dibenzyl chlorophosphite resulted in the expected dibenzyl ester-indole derivative 19. Catalytic hydrogenolysis of the benzyl oxygen bonds of 19 with hydrogen and palladized carbon followed by reaction with sodium methoxide in methanol as previously described (Pettit et al., Anticancer Drug. Des., 1998), resulted in the desired disodium phosphate indole prodrug 20 with an overall yield of 65%.

i) 2-(3′-tert-Butyldimethylsilyloxy-4′-methoxyphenyl)-6-methoxyindole (16)

To a boiling mixture of m-anisidine (0.72 g, 5.85 mmol) and N, N-dimethylaniline (2.4 mL) was added a solution of 15 (1.0 g, 2.78 mmol) in EtOAc (4.0 mL), slowly with a syringe. The reaction mixture was maintained at a temperature of 170° C. for 12 hours. Water was added, the phases separated and the aqueous layer extracted with EtOAc (3×25 mL). The combined organic layers were dried over Na₂SO₄ and concentrated under reduced pressure. Purification by flash column chromatography (silica gel, hexanes/EtOAc: 95/5) yielded 0.61 g (1.6 mmol, 57%) of the phenylindole 16 as white crystals.

¹H-NMR (300 MHz, CDCl₃): δ 8.11 (br s, 1H), 7.46 (d, J=8.5 Hz, 1H), 7.16 (dd, J=2.2, 8.3 Hz, 1H), 7.12 (d, J=2.2 Hz, 1H), 6.89 (d, J=8.3 Hz, 1H), 6.88 (d, J=1.9 Hz, 1H), 6.78 (dd, J=2.2, 8.6 Hz, 1H), 6.6 (d, J=2.0 Hz, 1H), 3.85 (s, 3H), 3.84 (s, 3H), 1.02 (s, 9H), 0.19 (s, 6H). ¹³C-NMR (75 MHz, CDCl₃): δ 156.4, 150.6, 145.4, 137.4, 136.9, 125.8, 123.7, 120.9, 118.2, 117.8, 112.4, 109.9, 98.7, 94.5, 55.7, 55.5, 25.7, 18.5, −4.6. Anal. Calcd for C₂₂H₂₉O₃NSi: C,68.89; H,7.62; N, 3.65. Found: C, 68.68; H, 7.60; N, 3.62.

ii) 2-(3′-tert-Butyldimethylsiloxy-4′-methoxyphenyl)-3-(3″,4″,5″-trimethoxybenzoyl)-6-methoxyindole (17)

To a well-stirred solution of 16 (0.511 g, 1.33 mmol) in o-dichlorobenzene (10 mL) was added 3,4,5-trimethoxybenzoylchloride (0.464 g, 2.01 mmol). The reaction mixture was heated to reflux at 160° C. for 12 hours. Then excess of o-dichlorobenzene was distilled off under reduced pressure and the resulting dark colored solid was subjected to flash chromatography (silica gel, 20% EtOAc in hexanes) to afford compound 17 as yellow oil. Recrystallization from hexanes-EtOAc afforded benzoyl indole derivative 17 as prismatic, pale-yellow crystals (0.355 g, 0.614 mmol, 46%) mp=165-167° C.

¹H NMR (300 MHz, CDCl₃): δ 8.39 (br s, 1H), 7.95 (d, J=9.9 Hz, 1H), 6.99 (s, 2H), δ 6.9 (m, 3H), δ 6.77 (d, J=2.3 Hz, 1H), 6.71 (d, J=8.8 Hz, 1H), 3.87 (s, 3H), 3.79 (s, 3H), 3.75 (s, 3H), 3.69 (s, 6H), 0.93 (s, 9H), 0.035 (s, 3H). ¹³C-NMR (75 MHz, CDCl₃): δ 191.8, 171.3, 157.2, 152.4, 151.5, 144.9, 142.1, 141.1, 136.4, 134.5, 125.0, 123.2, 122.4, 122.2, 121.8, 112.6, 111.5, 107.2, 94.5, 60.7, 55.9, 55.6, 55.3, 25.6, 18.3, −4.8. IR (Nujol, cm⁻¹): 3304, 3000, 2935, 2857, 2836, 1625, 1578, 1503, 1455, 1310, 1247, 1175, 1128. Anal. Calcd for C₃₂H₃₉O₇NSi: C,66.52; H,6.80; N, 2.42. Found: C, 66.51; H, 6.92; N, 2.29.

iii) 2-(3′-Hydroxy-4′-methoxyphenyl)-3-(3″,4″,5″-trimethoxybenzoyl)-6-methoxyindole (18)

To a well-stirred solution of 17 (0.29 g, 0.50 mmol) in CH₂Cl₂ (10 mL), under argon, was added tetrabutylammoniumfluoride solution (0.75 mL, 0.75 mmol, 1M in THF) at 0° C. The reaction mixture was stirred for 30 minutes allowing it to reach room temperature. Then, water (5 mL) was added, the phases separated and the aqueous phase was extracted with EtOAc (3×10 mL). The combined organic phases were dried over Na₂SO₄ and concentrated under reduced pressure. Purification by flash column chromatography (silica gel, hexanes/EtOAc: 60/40) afforded 0.20 g (0.43 mmol, 86%) of the desired indole phenol ligand 18 as a yellow powder.

¹H-NMR (300 MHz, CDCl₃): δ 8.53 (br s, 1H), 7.92 (d, J=9.5 Hz, 1H), 6.95 (s, 2H), 6.92 (dd, J=2.3, 6.7 Hz, 1H), 6.91 (m, 2H), 6.75 (dd, J=2.1, 8.3 Hz, 1H), 6.61 (d, J=8.4 Hz, 1H), 3.87 (s, 3H), 3.81 (s, 3H), 3.79 (s, 3H), 3.7(s, 6H). ¹³C-NMR (75 MHz, CDCl₃): δ 192.1, 157.3, 152.5, 146.9, 145.5, 142.4, 140.9, 136.3, 134.9, 125.2, 122.9, 122.4, 121.7, 114.6, 112.9, 111.6, 110.3, 107.1, 94.5, 60.8, 56.0, 55.9, 55.7. Anal. Calcd for C₂₆H₂₅O₇N: C, 67.38; H, 5.44; N, 3.02. Found: C, 67.13; H, 5.58; N, 2.90.

iv) 2-(3′-Dibenzylphosphate-4′-methoxyphenyl)-3-(3″, 4″,5″-trimethoxybenzoyl)-6-methoxyindole (19)

To a well-stirred solution of 18 (0.485 g, 1.05 mmol) under argon in acetonitrile (15 mL) was added CCl₄ (0.89 mL, 9.21 mmol) at −25° C. After stirring for 10 minutes, ethyldiisopropylamine (0.38 mL, 2.19 mmol) and N,N-dimethylaminopyridine (0.01 g, 0.105 mmol) were added. After 5 minutes, dibenzylphosphite (0.35 mL, 1.58 mmol) was added and the reaction mixture was stirred for 2 hours, allowing it to reach room temperature. At this point, a solution of KH₂PO₄ (25 mL, 0.5 M solution) was added and the product isolated by extraction with EtOAc (3×25 mL). The combined organic phases were dried over Na₂SO₄ and concentrated in vacuo to colorless oil. Purification by flash column chromatography (silica gel, hexanes/EtOAc: 40/60) yielded 0.639 g (0.883 mmol, 84%) of the desired product 19 as yellow powder.

¹H-NMR (300 MHz, CDCl₃): δ 10.29 (s, 1H), 7.75 (d, J=8.8 Hz, 1H), 7.34 (m, 10H), 7.14 (m, 1H), 6.92 (d, J=2.2 Hz, 1H), 6.89 (s, 2H), 6.81 (dd, J=2.4, 8.8 Hz, 2H), 6.24 (d, J=8.5 Hz, 1H), 5.17 (d, J=7.7 Hz, 4H), 3.77 (s, 3H), 3.7 (s, 3H), 3.6 (s, 6H), 3.27 (s, 3H). ¹³C-NMR (75 MHz, CDCl₃): δ 191.9, 171.2, 157.1, 152.5, 150.5, 141.1, 138.8, 136.7, 135.4, 128.6, 127.7, 124.5, 122.9, 122.1, 121.3, 112.6, 111.8, 111.5, 107.2, 94.7, 70.2, 70.1, 60.4, 55.9, 55.6, 55.5. ³¹p (120 MHz, CDCl₃): δ 6.02

v) 2-(3′-Disodiumphosphate-4′-methoxyphenyl)-3-(3″,4″,5″-trimethoxybenzoyl)-6-methoxyindole (20)

To a solution of dibenzyl ester 19 (0.639 g, 0.883 mmol) in 100% ethanol (10 mL) under argon, was added 5% palladium-carbon (0.30 g). The flask was then closed and the air was evacuated by aspirator vacuum. Then, H₂ gas was passed into the solution from a balloon. The reaction proceeded for about 30 minutes and TLC analysis (silica gel, hexanes/EtOAc: 30/70) showed no starting material. The reaction mixture was then filtered and concentrated under reduced pressure to give greenish-yellow foam.

To a well-stirred solution of the crude phosphate (0.611 g, 1.12 mmol) in dry methanol (10 mL) was added sodium methoxide (0.51 mL, 2.25 mmol, 4.37M solution) and the reaction mixture was stirred at room temperature for 12 hours. The methanol was removed in vacuo and the solid recrystallized from water-acetone to give 20 as a yellow colored crystalline solid (0.416 g, 0.71 mmol, 63%).

¹H-NMR (300 MHz, D₂O) δ 7.88 (d, J=8.7 Hz, 1H), 7.62 (m, 1H), 7.05 (d, J=2.2 Hz, 1H), 6.88 (dd, J=2.3, 8.8 Hz, 1H), 6.76 (s, 2H), 6.46 (d, J=8.6 Hz, 1H), 6.41 (dd, J=1.8, 8.6 Hz, 1H), 3.89 (s, 3H), 3.71 (s, 3H), 3.67 (s, 6H), 3.66 (s, 3H). ¹³C-NMR (75 MHz, D₂O): 195.4, 156.4, 151.7, 150.5, 150.4, 147.9, 142.8, 139.7, 136.6, 135.5, 125.8, 123.8, 122.2, 121.5, 120.5, 111.8, 111.2, 107.9, 95.5, 60.8, 56.1, 55.8, 55.7. ³¹P (120 MHz, D₂O): δ 0.69.

### Example 3

### Synthesis of Amine Functionalized 2-Aryl, 3-Aroyl Substituted Indoles and Corresponding Phosphoramidate Prodrugs

In addition to the phosphate ester prodrugs that are described in this application for indole-based anti-mitotic agents, we have also discovered that phosphorous based prodrug derivatives of the nitrogenated indoles may have therapeutic advantages as selective tumor vasculature destruction agents. These compounds are primarily serinamides, phosphoramidates, and related phosphate dianions that are assembled on the amino substituent of tubulin binding indole analogs of CA4. When utilized iii vivo, phosphoramidate analogs are able to provide a more soluble compound than the corresponding amine, thereby increasing the bioavailability of the parent drug. The P—N bond can be enzymatically cleaved by serum phosphatases releasing the amine which can inhibit tubulin assembly in a manner analogous to CA4P.

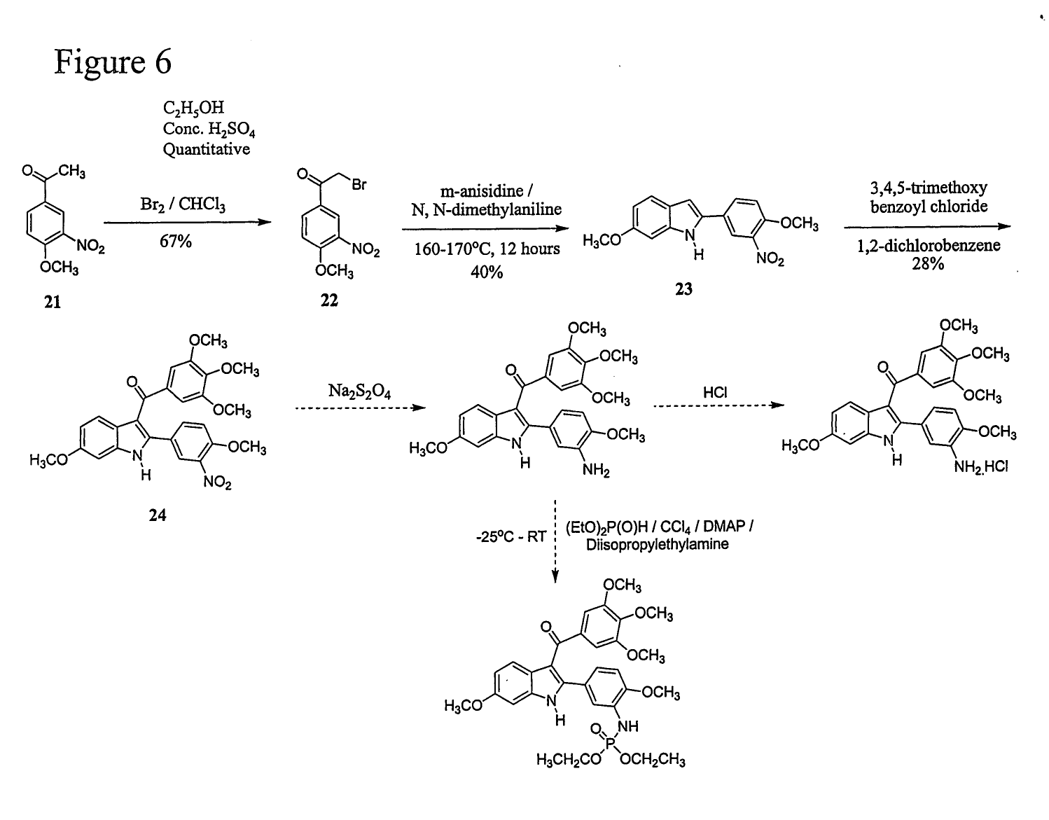

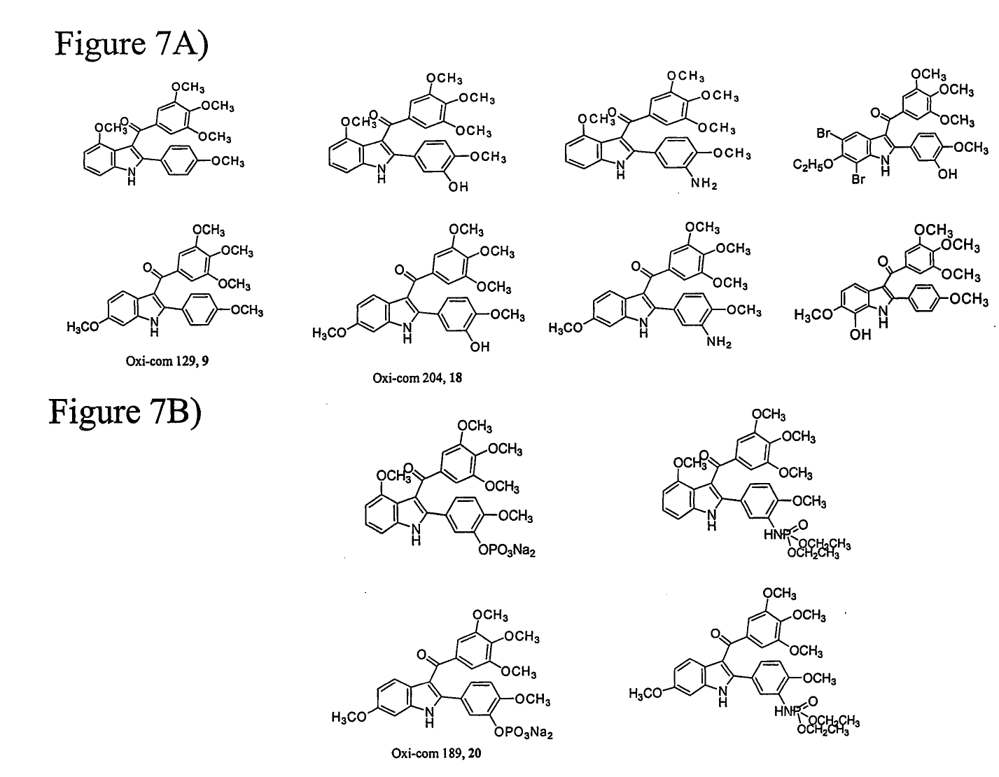

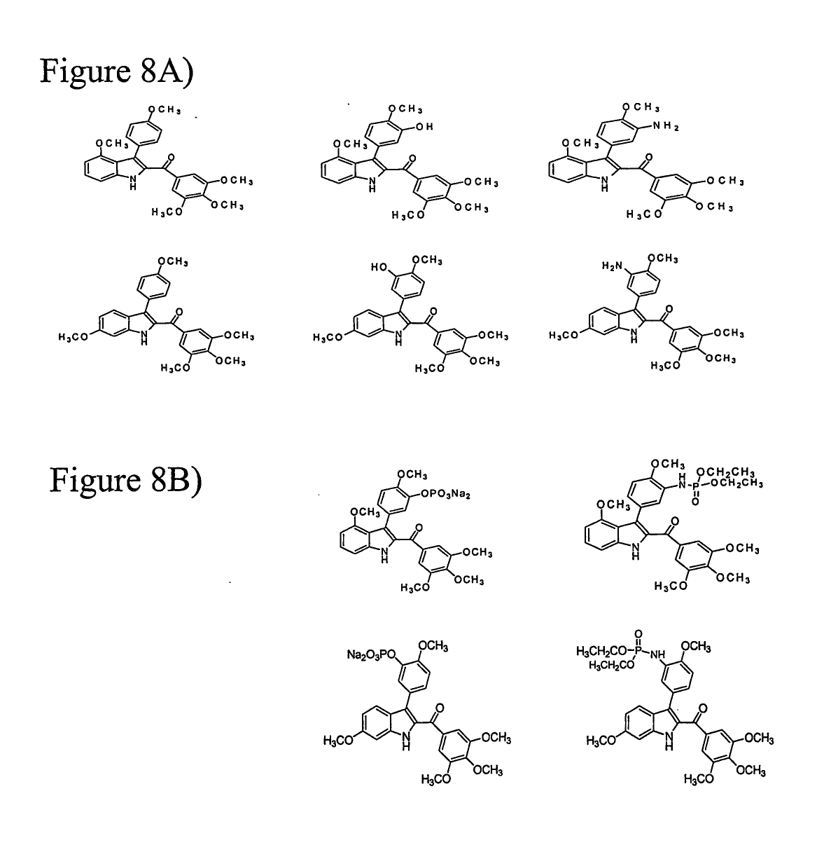

The amine-based prodrugs of the invention can be synthesized according to the scheme provided in FIG. 6.

A) 2-Aryl, 3-Aroyl Indole Phosphoramidate Prodrug

i) 4′-Methoxy-3′-nitro-2-bromacetophenone (22):

To a well-stirred solution of 3-Nitro-4-methoxyacetophenone 21 (5.334 g, 27.33 mmol) in chloroform (25 mL) at room temperature, was added a solution of bromine (1.40 mL, 27.33 mmol, 1.0 equivalent) in chloroform (5 mL), slowly dropwise. The reaction mixture was stirred for 30 minutes, periodically checking by TLC. Then water (25 mL) was added, organic layer was separated and the aqueous layer was extracted with CHCl₃ (3×15 mL). The combined organic phases were dried over anhydrous Na₂SO₄, filtered and rotovaped to afford a white-colored solid. This crude product was then recrystallized from hot ethanol (40 mL) to yield the desired bromoacetophenone derivative 22 as cream-colored, needle-shaped crystals (5.038 g, 18.38 mmol, 67%), R_{f}=0.286 (EtOAc/hexanes: 40/60).

During this procedure a small amount of 4′-methoxy-3′-nitro-2,2-dibromoacetophenone is also formed, R_{f}=0.404 (EtOAc/hexanes: 40/60). When the reaction was continued further beyond 30 minutes, it was observed that the monobromo derivative got converted to the dibromo derivative.

¹H NMR (300 MHz, CDCl₃): δ 8.48 (1H, d, J=2.3 Hz), 8.21 (1H, dd, J=2.2 Hz, J=8.8 Hz), 7.20 (1H, d, J=8.9 Hz), 4.40 (2H, s), 4.07 (3H, s).

¹³C NMR(75 MHz, CDCl₃): δ 188.45, 162.25, 156.68, 134.82, 126.76, 126.13, 113.55, 57.00, 29.89.

GC-MS: Retention time=14.36 minutes (120° C. at the rate of 10° C./minute).

ii) 2-(4′-Methoxy-3′-nitrophenyl)-6-methoxyindole (23):

To a well-stirred solution of meta-anisidine (2.6 mL, 23.24 mmol, 3.3 equivalents) in N, N-dimethylaniline (10 mL) at 150° C., was added a hot solution (kept at 60° C.) of 22 (1.93 g, 7.042 mmol) in ethanol (˜5 mL). The reaction mixture was stirred at 160-170° C. for 12 hours, periodically examining by TLC. Water was added when a dark colored solid separated inside the reaction flask. This solid dissolved on addition of EtOAc. Then, the phases separated and the aqueous layer was extracted with EtOAc (3×30 mL). The combined organic layers were dried over Na₂SO₄ and concentrated under reduced pressure to get a dark-colored solid. This crude product was further purified by recrystallization from EtOAc, to afford the desired product 23 as dark orange-red colored crystals (0.847 g, 2.84 mmol, 40%), R_{f}=0.333 (EtOAc/hexanes: 40/60).

¹H NMR (300 MHz, CDCl₃): δ10.63 (1H, s), 8.21 (1H, d, J=2.3 Hz), 8.06 (1H, dd, J=2.3 Hz, J=8.8 Hz), 7.44 (1H, d, J=8.6 Hz), 7.42 (1H, d, J=8.9 Hz), 6.93 (1H, d, J=2.2 Hz), 6.93 (1H, s,), 6.71 (1H, dd, J=2.3 Hz, J=8.6 Hz), 4.02 (3H, s), 3.81 (3H, s).

¹³C NMR (75 MHz, CDCl₃): 157.86, 152.10, 141.38, 139.43, 135.32, 130.80, 126.87, 124.39, 121.86, 121.43, 115.62, 111.07, 100.52, 95.21, 57.23, 55.70.

iii) 2-(4′-Methoxy-3′-nitrophenyl)-3-(3″,4″,5″-trimethoxybenzoyl)-6-methoxyindole (24):

To a well-stirred solution of 23 (0.592 g, 1.985 mmol) in ortho-dichlorobenzene (15 mL) at 150° C. was added 3,4,5-trimethoxybenzoyl chloride (0.782 g, 3.394 mmol, 1.71 equivalents) in portions. The reaction mixture was stirred at 160-170° C. for 10 hours and examined periodically by TLC. Then, it was allowed to cool to room temperature when a dark-colored solid was separated. This solid was filtered and a saturated solution of NaHCO₃ (20 mL) was added to the filtrate. Then, the phases separated and the aqueous layer extracted with CH₂Cl₂ (2×20 mL) and EtOAc (2×20 mL). The combined organic layers were dried over Na₂SO₄ and concentrated under reduced pressure. This crude mixture was subjected to flash chromatography (silica gel, EtOAc/hexanes: 50/50) and the desired product was isolated as a greenish-yellow solid.

The filtered solid was further purified by recrystallization from EtOAc-hexanes and was characterized to be the desired product. The combined weight (both from recrystallization and the column) of the product 24 was found to be 0.273 g (0.554 mmol, 28%), R_{f}=0.171 (EtOAc/hexanes: 50/50).

¹H NMR (300 MHz, CD₃OD): δ 11.10 (1H, s), 7.91 (1H, d, J=8.8 Hz), 7.69 (1H, d, J=2.2 Hz), 7.61 (1H, dd, J=2.3 Hz, J=8.7 Hz), 7.17 (1H, d, J=8.7 Hz), 6.99 (1H, d, J=2.0 Hz), 6.88 (1H, dd, J=2.3 Hz, J=8.8 Hz), 6.80 (2H, s), 3.90 (3H, s), 3.86 (3H, s), 3.68 (6H, s), 3.67 (3H, s).

¹³C NMR (75 MHz, CD₃OD): δ 194.58, 159.12, 154.05, 153.82, 143.40, 142.09, 140.46, 138.64, 136.89, 135.73, 127.63, 126.19, 123.86, 123.23, 114.81, 114.49, 113.27, 108.24, 95.59, 61.12, 57.37, 56.55, 56.02.

### Example 4

### Synthesis of N-Substituted Indoles

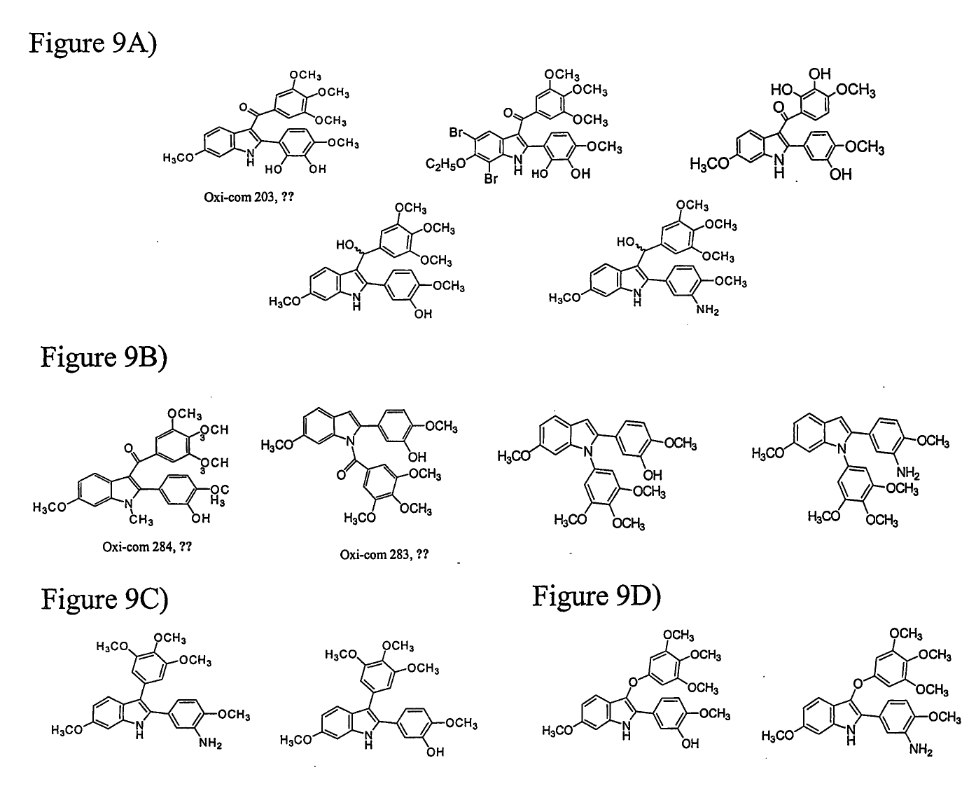

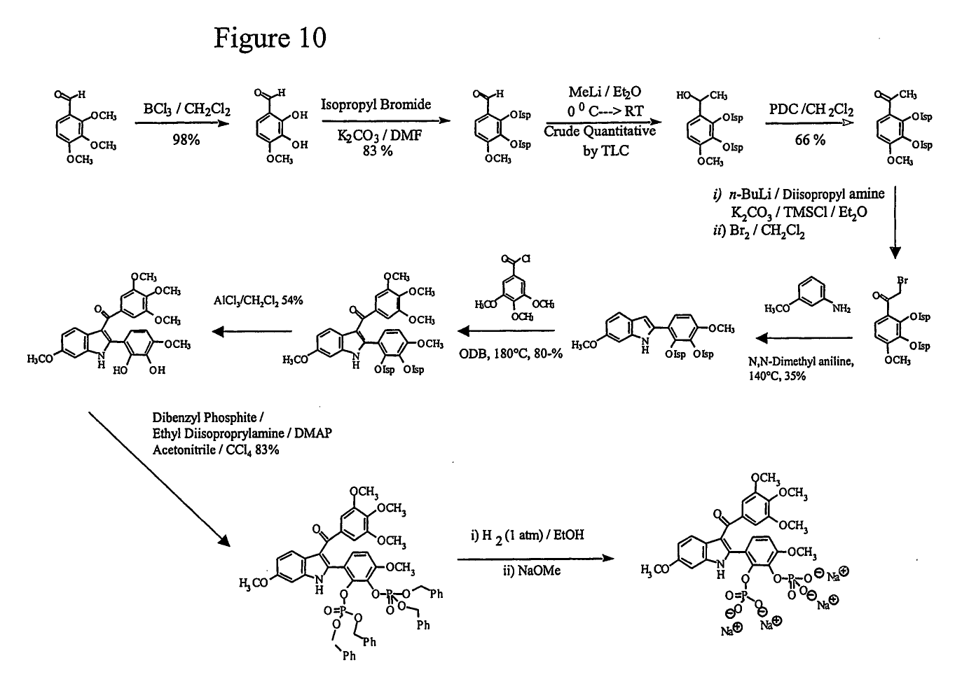

In addition to the 1H-Indole-based anti-mitotic agents described in this application, we have discovered that the nitrogen of the above indoles can be substituted with retention of tubulin binding activity. Similarly, the 2,3-diaroyl or diaryl substitution pattern of the indole ring can be shifted to a 1(N), 2 disubstitution pattern while retaining activity. Exemplary N-substituted diar(o)yl indoles are depicted in FIG. 9B. These compounds may be prepared by an addition elimination reaction utilizing the trimethoxyphenolic anion as a nucleophile as described by us for the benzo[b]thiophene compounds. Other linkage atoms between the aryl rings are conceivable as well.

A) Synthesis of an N-methyl, 2-aryl, 3-aroyl Indole Phosphate

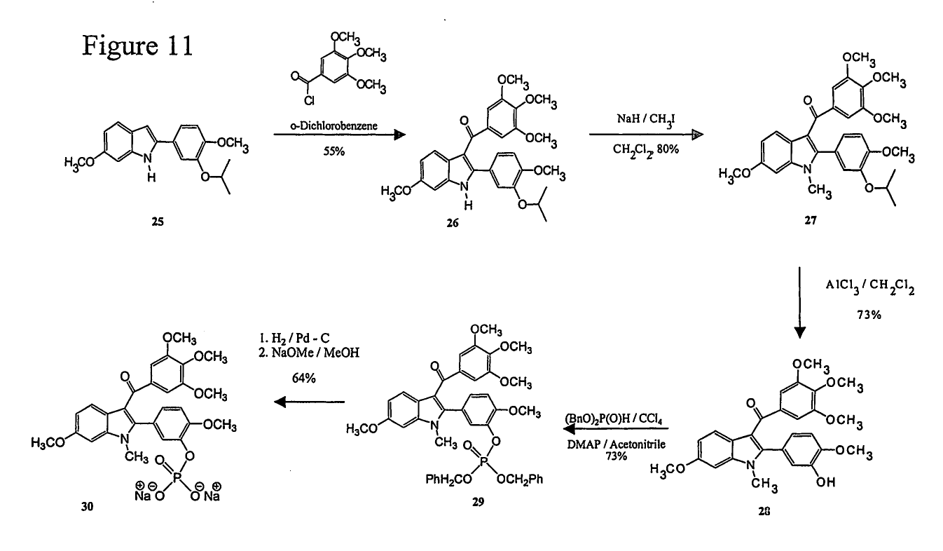

An N-methyl derivate of Oxi8007 was prepared as illustrated in FIG. 11.

i) 2-(3′-Isopropoxy-4′-methoxyphenyl)-3-(3″,4″,5″-trimethoxybenzoyl)-6-methoxy indole (26):

To a well-stirred solution of 2-(3′-isopropoxy-4′-methoxyphenyl)-6-methoxy indole 25 (1.074 g, 3.45 mmol) in o-dichlorobenzene (20 mL) was added 3,4,5-trimethoxybenzoylchloride (1.39 g, 6.036 mmol, 1.75 equivalents). The reaction mixture was heated to reflux at 160° C. for 16 hours and periodically examined by TLC. Then, excess o-dichlorobenzene was distilled off under reduced pressure and the resulting dark-colored solid was subjected to flash chromatography (silica gel, EtOAc/hexanes: 50/50) to isolate the desired product 26 (0.96 g, 1.90 mmol, 55%) as a yellow colored solid, R_{f}=0.286 (EtOAc/hexanes: 50/50).

¹H NMR (300 MHz, CDCl₃): δ 8.79 (1H, s), 7.94 (1H, d, J=9.4 Hz), 6.99 (1H, dd, J=2.1 Hz, J=8.2 Hz), 6.98 (2H, s), 6.92 (1H, dd, J=2.3 Hz, J=6.1 Hz), 6.90 (1H, d, J=2.3 Hz), 6.74 (1H, d, J=2.1 Hz), 6.73 (1H, d, J=8.4 Hz), 4.16 (1H, m), 3.85 (3H, s), 3.79 (3H, s), 3.78 (3H, s), 3.66 (6H, s), 1.21 (3H, d, J=5.6 Hz), 1.19 (3H, d, J=5.6 Hz).

¹³C NMR (75 MHz, CDCl₃): δ 191.78, 157.27, 152.46, 150.88, 147.10, 142.32, 141.17, 136.36, 134.52, 124.84, 123.18, 122.39, 120.96, 117.27, 112.57, 111.56, 111.51, 107.23, 94.52, 71.74, 64.43, 60.79, 55.89, 55.65, 25.32, 21.90.

Anal. Calcd for C₂₉H₃₁O₇N: C, 68.89; H, 6.18; N, 2.77. Found: C, 68.60; H, 6.20; N, 2.71.

ii) 2-(3′-Isopropoxy-4′-methoxyphenyl)-3-(3″,4″,5″-trimethoxybenzoyl)-N-methyl-6-methoxyindole (27):

To a well-stirred, ice-cooled solution of 26 (0.52 g, 1.03 mmol) in anhydrous CH₂Cl₂ (20 mL) was added sodium hydride (0.04 g, 1.75 mmol, 1.71 equivalents) slowly and carefully. The solution was stirred for 5 minutes and then methyl iodide (0.13 mL; 2.16 mmol, 2.1 equivalents) was added dropwise. The ice bath was removed and the reaction mixture was allowed to warm to room temperature slowly. It was then stirred at room temperature for 19 hours and was checked periodically by TLC. Water (15 mL) was added, organic layer was separated and the aqueous layer was extracted with CH₂Cl₂ (2×15 mL). The combined organic phases were dried over anhydrous Na₂SO₄, filtered and rotovaped. Purification by flash column chromatography (silica gel, EtOAc/hexanes: 30/70) afforded the pure product 27 as a white solid (0.426 g, 0.82 mmol, 80%), R_{f}=0.178 (EtOAc/hexanes: 40/60).

¹H NMR (300 MHz, CDCl₃): δ 8.00 (1H, d, J=8.7 Hz), 6.97 (1H, dd, J=2.3 Hz, J=8.7 Hz), 6.88 (1H, dd, J=1.9 Hz, J=7.9 Hz), 6.87 (1H, d, J=2.4 Hz), 6.84 (2H, s), 6.81 (1H, d, J=8.3 Hz), 6.66 (1H, d, J=1.9 Hz), 4.28 (1H, m), 3.93 (3H, s), 3.82 (3H, s), 3.78 (3H, s), 3.73 (6H, s), 3.66 (3H, s), 1.27 (6H, d, J=6.1 Hz).

¹³C NMR (75 MHz, CDCl₃): 191.82, 157.18, 152.17, 150.59, 146.75, 145.18, 140.53, 138.12, 135.32, 123.47, 123.31, 122.54, 121.82, 118.42, 114.02, 111.49, 111.17, 106.67, 93.56, 71.45, 60.69, 55.90, 55.86, 55.74, 31.41, 21.94.

Anal. Calcd for C₃₀H₃₃O₇N: C, 69.35; H, 6.40; N, 2.69. Found: C, 69.32; H, 6.44; N, 2.71.

iii) 2-(3′-Hydroxy-4′-methoxyphenyl)-3-(3″,4″,5″-trimethoxybenzoyl)-N-methyl-6-methoxyindole (28):

To a well-stirred, ice-cooled solution of 27 (0.426 g, 0.819 mmol) in anhydrous CH₂Cl₂ (15 mL) was added AlCl₃ (0.328 g, 2.46 mmol, 3.0 equivalents). The ice bath was removed and the solution was allowed to warm to room temperature. The reaction mixture was stirred at room temperature for 2 hours. Examination by TLC indicated the presence of starting material. Hence, added an additional 3 equivalents (0.328 g, 2.46 mmol) of AlCl₃ and then the reaction was stirred for another 1.5 hours when, examination by TLC indicated the completion of the reaction. Water (15 mL) was added, the organic phase was separated, and the aqueous phase was extracted with CH₂Cl₂ (2×10 mL). The combined organic phases were dried over anhydrous Na₂SO₄, filtered and rotovaped. Purification by flash column chromatography (silica gel, EtOAc/hexanes: 40/60) afforded the pure product 28 as a white solid (0.286 g, 0.599 mmol, 73%), R_{f}=0.167 (EtOAc/hexanes: 50/50).

¹H NMR (300 MHz, CDCl₃): δ 8.02 (1H, d, J=8.7 Hz), 6.96 (1H, dd, J=2.2 Hz, J=8.7 Hz), 6.86 (1H, d, J=2.1 Hz), 6.79 (1H, d, J=8.0 Hz), 6.78 (2H, s), 6.65 (2H, m), 5.77 (1H, s), 3.92 (3H, s), 3.82 (3H, s), 3.77 (3H, s), 3.74 (6H, s), 3.65 (3H, s).

¹³C NMR (75 MHz, CDCl₃): 192.02, 157.15, 152.14, 146.8, 145.28, 145.02, 140.08, 138.03, 135.67, 123.79, 123.39, 122.56, 121.72, 116.81, 114.19, 111.51, 110.06, 106.47, 93.53, 60.58, 55.92, 55.86, 55.72, 31.29.

Anal. Calcd for C₂₇H₂₇O₇N: C, 67.91; H, 5.69; N, 2.93. Found: C, 67.18; H, 5.97; N, 2.86.

iv) 2-(3′-Dibenzylphosphate-4′-methoxyphenyl)-3-(3″,4″,5″-trimethoxybenzoyl)-N-methyl-6-methoxyindole (29):

To a well-stirred solution of 28 (0.312 g, 0.653 mmol) under argon in acetonitrile (15 mL) was added CCl₄ (0.56 mL, 5.75 mmol, 8.8 equivalents) at −25° C. After stirring for 10 minutes ethyldiisopropylamine (0.24 mL, 1.37 mmol, 2.1 equivalents) and N,N-dimethylaminopyridine (0.008 g, 0.06 mmol, 0.1 equivalents) were added. Further, after 5 minutes, dibenzylphosphite (0.22 mL, 0.987 mmol, 1.51 equivalents) was added and the reaction mixture was stirred for 1.5 hours, allowing it to reach the room temperature and periodically examining by TLC. At this point, a solution of KH₂PO₄ (20 mL, 0.5 M aqueous solution) was added and the product was isolated by extraction with EtOAc (3×50 mL). The combined organic phases were dried over Na₂SO₄ and concentrated in vacuo. Purification by flash column chromatography (silica gel, EtOAc/hexanes: 50/50) yielded 0.35 g (0.474 mmol, 73%) of the desired product 29 as a white solid, R_{f}=0.524 (EtOAc/hexanes: 70/30).

¹H NMR (300 MHz, CDCl₃): δ 7.88 (1H, d, J=8.7 Hz), 7.31 (10H, m), 7.17 (1H, m), 7.03 (1H, m), 6.93 (1H, dd, J=2.2 Hz, J=8.8 Hz), 6.84 (1H, d, J=2.2 Hz), 6.83 (2H, s), 6.78 (1H, d, J=8.5 Hz), 5.14 (4H, d, J=8.1 Hz), 3.91 (3H, s), 3.79 (3H, s), 3.76 (3H, s), 3.70 (6H, s), 3.54 (3H, s).

³¹P (120 MHz, CDCl₃): δ 5.95.

Anal. Calcd for C₄₁H₄₀O₁₀NP: C, 66.75; H, 5.46; N, 1.89. Found: C, 66.81; H, 5.53; N, 1.98

v) 2-(3′-Disodiumphosphate-4′-methoxyphenyl)-3-(3″,4″,5″-trimethoxybenzoyl)-N-methyl-6-methoxyindole (30):

To a solution of dibenzyl ester (0.32 g, 0.434 mmol) in 100% ethanol (10 mL) under argon, was added 5% palladium-carbon (0.10 g). The flask was then closed and the air was evacuated by aspirator vacuum. Then, H₂ gas was passed into the solution from a balloon. The reaction proceeded for about 30 minutes and TLC analysis (silica gel, EtOAc/hexanes: 70/30) showed no starting material. The reaction mixture was then filtered through celite and concentrated under reduced pressure to afford yellow foam.

To a well-stirred solution of the crude phosphate (0.223 g, 0.400 mmol) in dry methanol (15 mL) was added sodium methoxide (0.18 mL, 0.800 mmol, 4.37M solution in methanol, 2.0 equivalents) and the reaction mixture was stirred at room temperature for 12 hours. The methanol was removed in vacuo and the solid recrystallized from water-acetone to afford the desired salt 30 as a yellow-colored solid (0.168 g, 0.279 mmol, 64%).

¹H NMR (300 MHz, CD₃OD): δ 7.89 (1H, d, J=8.8 Hz), 7.79 (1H, m), 7.02 (1H, d, J=2.0 Hz), 6.88 (1H, dd, J=2.2 Hz, J=8.8 Hz), 6.76 (2H, s), 6.62 (1H, d, J=8.3 Hz), 6.58 (1H, dd, J=2.1 Hz, J=8.4 Hz), 3.90 (3H, s), 3.77 (3H, s), 3.76 (3H, s), 3.74 (6H, s), 3.69 (3H, s).

¹³C NMR (75 MHz, CD₃OD): 194.94, 158.8, 153.72, 152.24, 148.86, 145.63, 145.56, 141.52, 139.92, 137.36, 126.46, 124.31, 123.99, 123.16, 114.56, 112.86, 112.59, 107.97, 94.75, 61.10, 56.84, 56.62, 56.15, 32.37.

³¹P (120 MHz, CD₃OD): δ 1.84

B) Synthesis of an N-aroyl, 2-aryl Indole Phosphate

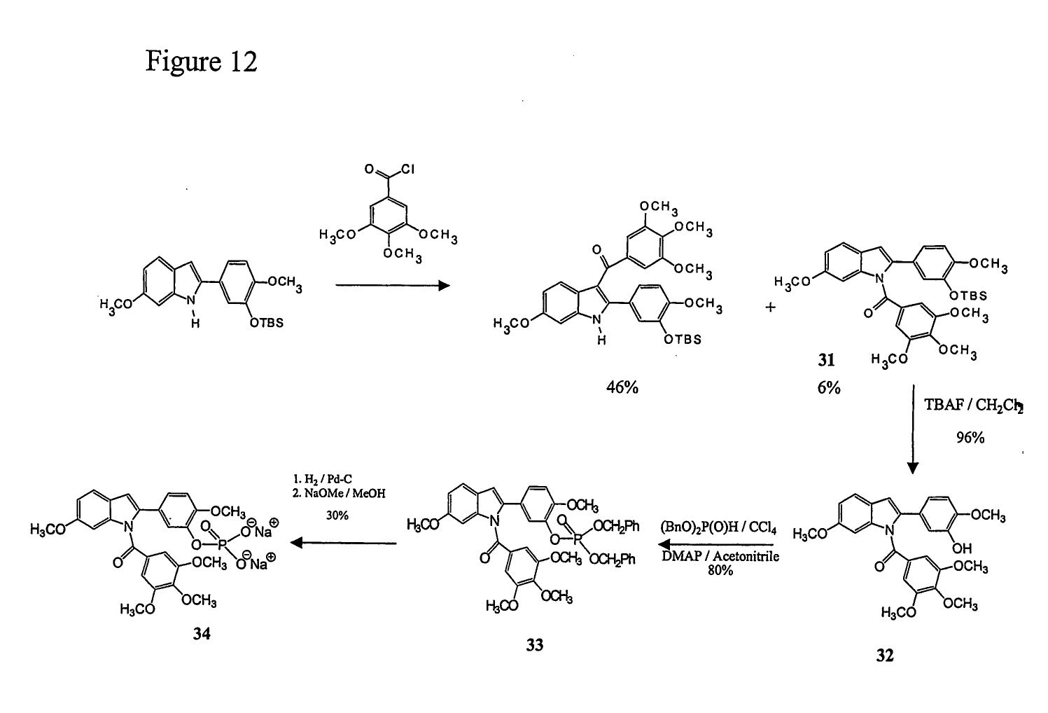

The N-aroyl, 2-aryl Indole Phosphate was prepared as illustrated in FIG. 12.

i) 2-(3′-tert-Butyldimethylsiloxy-4′-methoxyphenyl)-N-(3″,4″,5″-trimethoxybenzoyl)-6-methoxyindole (31):

To a well-stirred solution of 2-(3′-tert-butyldimethylsiloxy-4′-methoxyphenyl)-6-methoxyindole (0.218 g, 0.568 mmol) in o-dichlorobenzene (10 mL) was added 3,4,5-trimethoxybenzoylchloride (0.198 g, 0.858 mmol, 1.51 equivalents). The reaction mixture was heated to reflux at 160° C. for 12 hours. Then excess of o-dichlorobenzene was distilled off under reduced pressure and the resulting dark-colored solid was subjected to flash chromatography. The first product isolated was (silica gel, EtOAc/hexanes: 5/95) compound 31 as greenish-yellow solid (0.0196 g, 0.034 mmol, 6%), R_{f}=0.474 (EtOAc/hexanes: 40/60).

¹H NMR (300 MHz, CDCl₃): δ 7.47 (1H, d, J=8.5 Hz), 7.47 (1H, d, J=2.3 Hz), 6.91 (1H, dd, J=2.3 Hz, J=8.6 Hz), 6.85 (2H, s), 6.83 (1H, dd, J=2.2 Hz,

J=8.3 Hz), 6.71 (1H, d, J=2.2 Hz), 6.63 (1H, d, J=8.4 Hz), 6.60 (1H, s), 3.83 (3H, s), 3.78 (3H, s), 3.75 (6H, s), 3.70 (3H, s), 0.95 (9H, s), 0.055 (6H, s).

¹³C NMR (75 MHz, CDCl₃): δ 169.82, 157.67, 152.49, 150.27, 144.61, 142.00, 139.62, 139.10, 129.76, 126.25, 123.04, 121.36, 120.87, 120.56, 112.55, 111.59, 107.94, 107.88, 97.94, 60.77, 56.10, 55.61, 55.32, 25.61, 18.30, −4.79.

IR (Nujol, cm⁻¹): 3005, 2964, 2939, 2883, 2857, 2835, 1679, 1617, 1589, 1516, 1215.

Mass Spectrometry: (FAB) m/z 577.39

Anal. Calcd for C₃₂H₃₉O₇NSi: C, 66.52; H, 6.80; N, 2.42. Found: C, 66.66; H, 6.78; N, 2.42.

ii) 2-(3′-Hydroxy-4′-methoxyphenyl)-N-(3″,4″,5″-trimethoxybenzoyl)-6-methoxyindole (32):

To a well-stirred solution of 31 (0.185 g, 0.320 mmol) in CH₂Cl₂ (10 mL), under argon, was added tetrabutylammoniumfluoride solution (0.67 mL, 0.672 mmol, 1M in THF, 2.1 equivalents) at 0° C. The reaction mixture was stirred for 20 minutes allowing it to reach room temperature. Then water (10 mL) was added, the phases separated and the aqueous phase was extracted with EtOAc (3×10 mL). The combined organic phases were dried over Na₂SO₄ and concentrated under reduced pressure. Purification by flash column chromatography (silica gel, EtOAc/hexanes: 30/70) afforded 0.142 g (0.306 mmol, 96%) of the desired product 32 as a white powder, R_{f}=0.325 (EtOAc/hexanes: 50/50).

¹H NMR (300 MHz, CDCl₃): δ 7.50 (1H, d, J=2.2 Hz), 7.49 (1H, d, J=8.5 Hz), 6.92 (1H, dd, J=2.3 Hz, J=8.5 Hz), 6.84 (2H, s), 6.80 (1H, d, J=2.1 Hz), 6.73 (1H, dd,

J=2.2 Hz, J=8.3 Hz), 6.62 (1H, s), 6.61 (1H, d, J=8.3 Hz), 5.50 (1H, s), 3.84 (3H, s), 3.80 (3H, s), 3.78 (3H, s), 3.77 (6H, s).

¹³C NMR (75 MHz, CDCl₃): δ 169.75, 157.65, 152.44, 145.75, 145.02, 141.68, 139.59, 139.04, 130.02, 126.62, 122.97, 120.89, 119.82, 114.34, 112.53, 110.14, 108.17, 107.71, 98.12, 60.68, 56.09, 55.76, 55.57.

Anal. Calcd for C₂₆H₂₅O₇N: C, 67.38; H, 5.44; N, 3.02. Found: C, 67.30; H, 5.55; N, 3.08.

iii) 2-(3′-Dibenzylphosphate-4′-methoxyphenyl)-N-(3″,4″,5″-trimethoxybenzoyl)-6-methoxyindole (33):

To a well-stirred solution of 32 (0.165 g, 0.356 mmol) under argon in acetonitrile (10 mL) was added CCl₄ (0.3 mL, 3.13 mmol, 8.8 equivalents) at −25° C. After stirring for 10 minutes ethyldiisopropylamine (0.13 mL, 0.747 mmol, 2.1 equivalents) and N,N-dimethylaminopyridine (0.004 g, 0.036 mmol, 0.1 equivalents) were added. Further, after 5 minutes, dibenzylphosphite (0.12 mL, 0.537 mmol, 1.51 equivalents) was added and the reaction mixture was stirred for 2 hours, allowing it to reach the room temperature and periodically examining by TLC. At this point, a solution of KH₂PO₄ (10 mL, 0.5 M aqueous solution) was added and the product was isolated by extraction with EtOAc (3×25 mL). The combined organic phases were dried over Na₂SO₄ and concentrated in vacuo. Purification by flash column chromatography (silica gel, EtOAc/hexanes: 50/50) afforded 0.207 g (0.286 mmol, 80%) of the desired product 33, R_{f}=0.559 (EtOAc/hexanes: 60/40).

¹H NMR (300 MHz, CDCl₃): δ 7.48 (1H, d, J=8.6 Hz), 7.45 (1H, d, J=2.3 Hz) 7.31 (10H, m), 7.14 (1H, m), 6.99 (1H, m), 6.93 (1H, dd, J=2.3 Hz, J=8.6 Hz), 6.83 (2H, s), 6.68 (1H, d, J=8.6 Hz), 6.56 (1H, s), 5.12 (4H, d, J=8.2 Hz), 3.82 (3H, s), 3.76 (3H, s), 3.71 (6H, s), 3.67 (3H, s).

¹³C NMR (75 MHz, CDCl₃): δ 169.38, 157.70, 152.48, 149.59, 141.82, 139.07, 138.44, 135.48, 129.82, 128.38, 127.78, 126.07, 125.04, 122.82, 120.94, 120.80, 112.47, 112.05, 108.78, 107.66, 98.07, 69.77, 69.69, 60.59, 56.00, 55.67, 55.46.

³¹P (120 MHz, CDCl₃): δ −5.99.

Anal. Calcd for C₄₀H₃₈O₁₀NP: C, 66.38; H, 5.29; N, 1.93. Found: C, 66.09; H, 5.36; N, 2.04.

iv) 2-(3′-Disodiumphosphate-4′-methoxyphenyl)-N-(3″,4″,5″-trimethoxybenzoyl)-6-methoxyindole (34):

To a solution of dibenzyl ester 33 (0.197 g, 0.272 mmol) in 100% ethanol (15 mL) under argon, was added 5% palladium-carbon (0.10 g). The flask was then closed and the air was evacuated by aspirator vacuum. Then, H₂ gas was passed into the solution from a balloon. The reaction proceeded for about 30 minutes and TLC analysis (silica gel, EtOAc/hexanes: 60/40) showed no starting material. The reaction mixture was then filtered through a pad of celite and concentrated in vacuo.

To a well-stirred solution of the crude phosphate (0.144 g, 0.265 mmol) in dry methanol (5 mL) was added sodium methoxide (0.12 mL, 0.531 mmol, 4.37M solution in methanol, 2.0 equivalents) and the reaction mixture was stirred at room temperature for 12 hours. The methanol was removed in vacuo and the resulting solid was recrystallized from water-acetone to afford the desired salt 34 as an off-white solid (0.048 g, 0.082 mmol, 30%).

¹H NMR (300 MHz, CD₃OD): δ 7.51 (1H, s), 7.05 (1H, d, J=8.2 Hz), 6.77 (1H, d, J=8.4 Hz), 6.66 (2H, s), 6.63 (1H, d, J=2.4 Hz), 6.59 (1H, dd, J=2.1 Hz, J=8.3 Hz), 5.44 (1H, m), 3.78 (3H, s), 3.74 (6H, s), 3.61 (6H, s).

¹³C NMR (75 MHz, CD₃OD): 160.65, 154.41, 150.94, 150.85, 146.47, 145.07, 140.76, 137.38, 133.34, 126.57, 124.97, 119.16, 118.53, 112.74, 111.55, 105.69, 67.08, 61.17, 56.78, 56.61, 55.99.

³¹P (120 MHz, CD₃OD): δ 1.64

C) Synthesis of an 2,3-Diaryl-Dihydoxyindole and Corresponding Diphosphate Prodrug

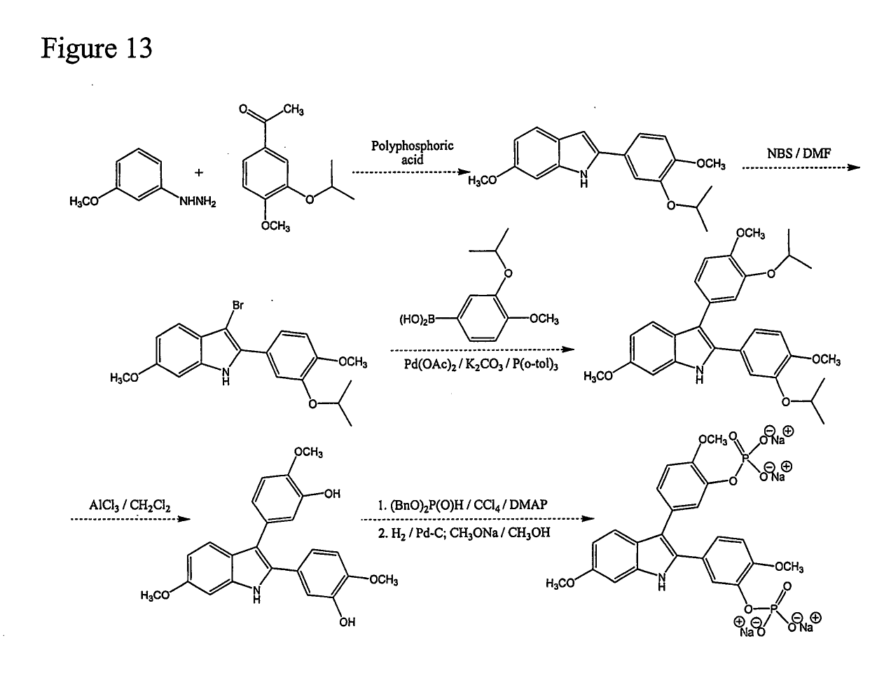

An exemplary 2,3-diaryl indoles will be prepared as illustrated in FIG. 13.

2-(3,4,5-trimethoxyphenyl)-6-methoxyindole will be prepared Fischer indole synthesis starting from 3-methoxyphenylhydrazine and 3,4,5-trimethoxyacetophenone using the methods described by Liu (Liu et al, J. Am. Chem. Soc., 2003). The resulting derivatized indole will then be reacted with N-bromosuccinimide in dimethylformamide to yield 3-bromo-2-(3,4,5-trimethoxyphenyl)-6-methoxyindole (Bunker et al, Bioorg. Med. Chem. Lett., 1996; Masanobu et al, Heterocycles, 1992). The product obtained will then be reacted (through a Suzuki coupling reaction) with 3-isopropoxy-4-methoxyphenylboronic acid to obtain the expected 2,3-diaryl substituted indole derivative (Liu et al, Tetrahedron Letters, 2000). The isopropyl group, in the resulting 2,3-diarylindole, will be removed by treatment with aluminum trichloride. The resulting hydroxy group will then be converted to the disodiumphosphate functionality leading to the prodrug.

### Example 5

### Synthesis of Indole Diaryl Ethers

In previous studies with the benzo[b]thiophene ligands, we have demonstrated that the carbonyl group can be replaced with an oxygen to generate a new compound which maintains the same or similar biological efficacy with tubulin. Similarly, the replacement of the carbonyl group in the described indole ligand may be replaced with an oxygen atom (ether linkage, —(C═O)—) to generate a new derivative which would be predicted to have good activity with tubulin (FIG. 9D). This compound may be prepared by an addition elimination reaction utilizing the trimethoxyphenolic anion as a nucleophile as described by us for the benzo[b]thiophene compounds. Other linkage atoms between the aryl rings are conceivable as well, including thioethers (—S—), secondary alcohols (—CH(OH)—, and methylenes (—CH2—). These compounds are intended to form a one-atom bridge between the substituted aryl and the indole ring. For example, the secondary alcohols can be created by reduction of corresponding ketones (—C═O)— with sodium borohydride, and methylenes can be created by reduction with trifluoroacetic acid. Alternatively, a single covalent bond can substitute for the 1-atom linker (see FIG. 9C).

### Example 6

### Inhibition of Tubulin Assembly

IC₅₀ values for tubulin assembly were determined according to a previously described procedure (Bai et al., Cancer Research, 1996). Purified tubulin is obtained from bovine brain cells as previously described (Hamel and Lin, Biochemistry, 1984). Various amounts of inhibitor were preincubated for 15 minutes at 37° C. with purified tubulin. After the incubation period, the reaction was cooled and GTP was added to induce tubulin assembly. Assembly was then monitored in a Gilford spectrophotometer at 350 nm. The final reaction mixtures (0.25 ml) contained 1.5 mg/ml tubulin, 0.6 mg/ml microtubule-associated proteins (MAPs), 0.5 mM GTP, 0.5 mlM MgCl₂, 4% DMSO and 0.1M 4-morpholineethanesulfonate buffer (MES, pH 6.4). IC₅₀ is the amount of inhibitor needed to inhibit tubulin assembly 50% with respect to the amount of inhibition that occurs in the absence of inhibitor.

TABLE 1
In Vitro Inhibition of Tubulin Assembly.

| Compound | IC₅₀ (μM) |
|---|---|
| CA-4 | 0.73 |
| BbT-H | 1.1 |
| BbF-H | 2.1 (pM) |
| Oxi-com 129 | 0.5-1 |
| Oxi-com 204 | 1-2 |
| Oxi-com 203 | 4 |

### Example 7

### In vitro Cytotoxicity Activity Against Cancer Cell Lines

Newly prepared compounds were evaluated for cytotoxic activity against a variety of cell lines derived from human tumors using an assay system similar to the National Cancer Institute procedure previously described (Monks et al, J. Natl. Cancer Inst., 1991). Briefly, the cell suspensions, diluted according to the particular cell type and the expected target cell density (5,000-40,000 cells per well based on cell growth characteristics), were added by pipet (100 μl) to 96-well microtiter plates. Inoculates were allowed a preincubation time of 24-28 hours at 37° C. for stabilization. Incubation with the inhibitor compounds lasted for 48 hours in 5% CO₂ atmosphere and 100% humidity. Determination of cell growth was performed by in situ fixation of cells, followed by staining with a protein-binding dye sulforhodamine B (SRB), which binds to the basic amino acids of cellular macromolecules. The solubilized stain was measured spectrophotometrically.

Several compounds were evaluated for cytotoxic activity against human P388 leukemia cell lines. The effective dose or ED₅₀ value (defined as the effective dosage required to inhibit 50% of cell growth) was measured. These and additional compounds were evaluated in terms of growth inhibitory activity against several other human cancer cell lines including: central nervous system (“CNS”, SF-295), pancreas (BXPC-3), non-small cell lung cancer (“lung-NSC”, NCI-H460), breast (MCF-7), colon (KM20L2), ovarian (OVCAR-3), and prostate (DU-145). The results are described in Table 2 below. The growth inhibition GI₅₀ (defined as the dosage required to inhibit tumor cell growth by 50% is listed for each cell line.

TABLE 2
In vitro Cytotoxicity against Human Cancer Cell Lines

| | ED50 (ug/ml) | | | | | | | |
|---|---|---|---|---|---|---|---|---|
| | for P-388 | GI₅₀ (μg/ml) for Cell Line | | | | | | |
| Compound | Cell Line | OVCAR-3 | SF-295 | BXPC-3 | NCI-H460 | MCF-7 | KM20L2 | DU-145 |
| BbT-H | | | | | | | | |
| BbF-H | | | | 0.038 | | | | 0.062 |
| Oxi-com 129 | 0.0133 | 0.0024 | 0.0024 | 0.0020 | 0.0026 | | 0.0017 | 0.0023 |
| Oxi-8007 | | | 0.0055 | 0.046 | 0.0027 | 0.0023 | 0.0043 | <0.001 |
| Oxi-com 283 | | | 3.0 | 4.2 | 3.4 | 0.35 | >10 | >10 |
| Oxi-com 284 | | | 0.33 | 1.6 | | 0.34 | 0.95 | 0.62 |

### Example 8

### Inhibition of Tumor Blood Flow

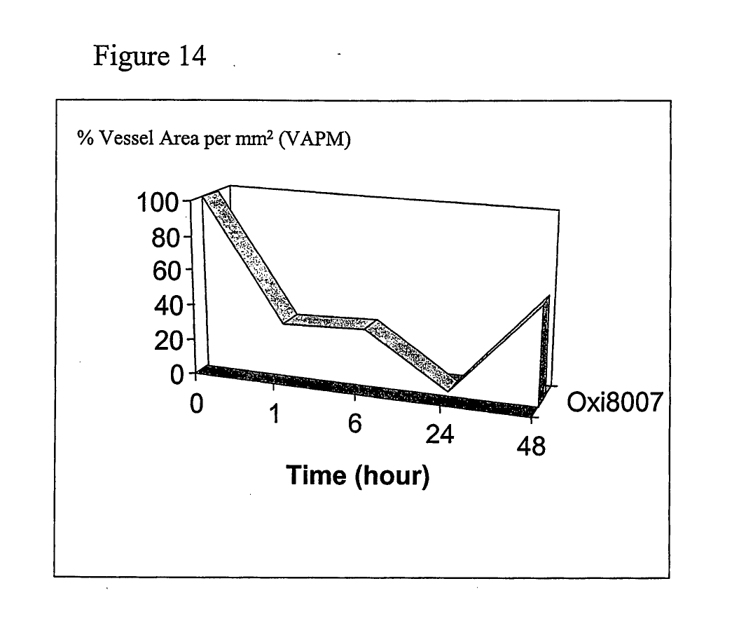

The antivascular effects of the indole phosphate prodrug Oxi-8007, was assessed in tumor-bearing mice using a Fluorescent Bead Assay. A MHEC-5T hemangioendothelioma tumor model was established by subcutaneous injection of 0.5×106 cultured transformed cell murine myocardial vascular endothelial cell line (“MHEC5-T”) cells into the right flank of Fox Chase CB-17 Severe Combined Immunodeficient (“SCID”) mice. When transplanted tumors reached a size of 500 mm³ (a size without development of necrosis), the mice received a single intraperitoneal (i.p.) injection of saline control or compound at doses ranging from 3.2 to 25 mg/kg. At 24 hours post-treatment, mice were injected intravenously with 0.25 ml of diluted FluoSphere beads (1:6 in physiological saline) in the tail vein, sacrificed after 3 minutes, and tumor was excised for cryosectioning. Tumor cryosections at a thickness of 8 um were directly examined using quantitative fluorescent microscopy. Blood vessels were indicated by blue fluorescence from injected beads. For quantification, image analysis of 3 sections from three tumors treated in each group were examined and vascular shutdown was expressed as vessel area (mm²) per tumor tissue area (mm²) as a percentage of the control (“%VAPM”). The results as shown in Table 4 indicate a clear dose-dependent effect of the agent on tumor blood flow as indicated by the reduction in blood vessel area at 24 hours following administration of Oxi8007. Administration of a dose of 100 mg/kg of Oxi-8007 or higher was particularly effective, causing a >98% reduction in tumor vessel volume relative to the control. A time-course of the effect of OX08007 on tumor blood flow is illustrated in FIG. 14.

TABLE 3
Vascular Targeting Activity of Oxi8007 prodrug

| Dose (mg/kg) | % VAPM |
|---|---|
| 0 | 100 |
| 50 | 52 |
| 100 | 2 |
| 200 | 1 |

Additional compounds of the present invention were tested for antivascular effects at two dosages (100 mg/kg and 10 mg/kg) using the same Fluorescent Bead Assay as in the previous experiment. The results are summarized in Table 4 below.

TABLE 4
Vascular Targeting Activity of N-substituted
Indole phosphate prodrug

| Compound | % VAPM at 100 mg/kg dose | % VAPM at 10 mg/kg dose |
|---|---|---|
| Oxi-com 285 | 74 | 30 |

### Example 9

### Evaluation of Tumor Growth Control in vivo

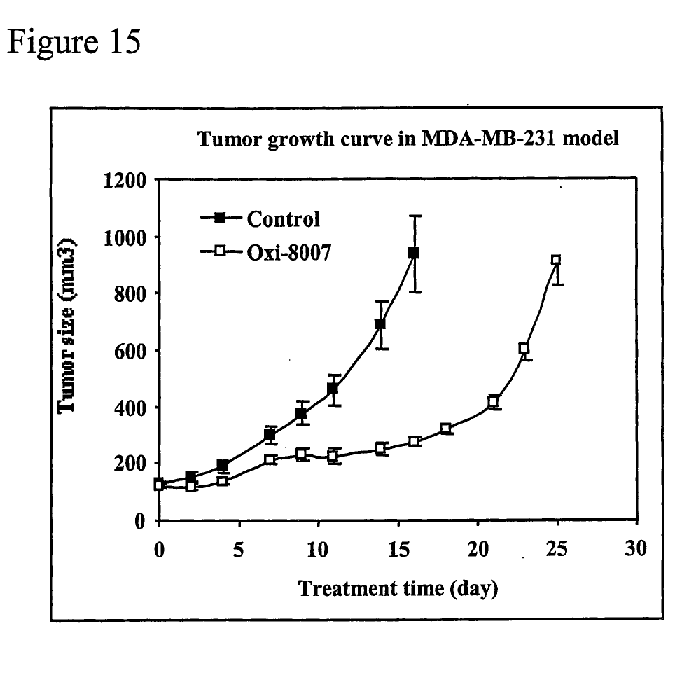

The antitumor activity of indole phosphate prodrug, Oxi-8007, was assessed in tumor-bearing mice by measuring its effects on tumor volume. A human breast adenocarcinoma model was established by subcutaneous injection of cultured MDA-MB-231 cells in Fox Chase CB-17 SCID mice. When the average tumor size reached 50-100 mm³, mice were randomly divided into several groups (n=10) with no significant difference in body weight and tumor size. Mice were administered Oxi-8007 in saline carrier at doses of 25, 50 or 100 mg/kg by daily intraperitoneal (i.p.) injection for 5 consecutive days (Q1×5). Saline carrier only was used as the control treatment. On Day 3, 7, 10, 13, 17 and 23, tumors were excised from animals in each treatment group (n=2) and measured by width and length. Tumor volume was calculated according to the following formula: Length×Width²×0.4. The dosage effects of Oxi-com 197 is illustrated in FIG. 15. Administration of X mg/kg doses of the drug significantly inhibited tumor growth relative to control treatment.

### ALTERNATIVE EMBODIMENTS

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

It should be readily apparent to any practitioner skilled in the art that there are various ways of appending trimethoxyaryl and trimethoxyaroyl groups around an indole molecular scaffold in a manner which will result in a similar molecular conformation capable of undergoing pseudo pi-pi stacking. In particular, the compounds of the subject invention can be synthesized in the alternative by previously described coupling methods such as the palladium-catalyzed coupling method of Flynn that involves multicomponent coupling of an o-iodoacetanilide and a methoxyphenylethyne in the presence of a palladium catalyst and methylmagnesium chloride to give an o-alkynylphenolate intermediate. Upon heating in a nitrogen or carbon monoxide atmosphere, the intermediate undergoes cyclization and reductive elimination in the presence of a methoxy-substituted aryl iodide to afford 2,3-diarylindole or 2-aryl, 3-aroyl indoles, respectively (Chaplin J H et al, Chem. Commun., 2001; Flynn et al, J. Med. Chem., 2002, PCT Pub. No. WO02/060872). Alternatively, the indole ligands of the invention can be obtained at room temperature in a one-pot reaction described by Cacchi, in which an o-iodotrifluoroacetanilide is coupled to the alkyne under Sonogashira conditions in acetonitrile to give an alkynylphenolate that, when treated with K₂CO₃ under the appropriate atmospheric conditions, affords the 2,3-diarylindole or 2-aryl,3-aroyl indoles of the invention (Arcadi et al, Tetrahedron Lett., 1992; Arcadi et al, Tetrahedron, 1994).

In addition, although the trimethoxyaryl motif seems optimal for enhanced tubulin binding, it is also very possible that another combination of alkoxy substituents (such as ethoxy, propoxy, isopropoxy, allyloxy, etc.) either as a trisubstituted pattern or as disubstituted (with one type of alkoxy moiety) and monosubstituted (with a different alkoxy moiety), or with three distinct types of alkoxy moieties may also have good tubulin binding characteristics. It is also conceivable that instead of having aryl alkoxy groups, it may be possible to substitute simply aryl-alkyl and aryl-alkenyl moieties and still maintain the enhanced cytotoxicity profile. Phenolic groups may also have activity on these described indole ligands. The synthesis of any of these modified indole-ligands will be very straightforward for anyone skilled in the art, and often will only involve a different choice of initial starting materials. To prepare these alternative ligands, the same synthetic schemes as disclosed herein or similar schemes with only slight modifications may be employed.

### REFERENCES

The following citations are incorporated in pertinent part by reference herein for the reasons cited.
(1) Arcadi A, Cacchi S, Marinelli F. A versatile approach to 2,3-disubstituted indoles through the palladium-catalyzed cyclization of o-alkynyltrifluoroacetanilides with vinyl triflates and aryl halides. Tetrahedron Lett. 1992, 33, 3915-8.
(2) Arcadi A, Cacchi S, Carnicelli V, Marinelli F. 2-substituted-3-acylindoles through the palladium-catalyzed carbonylate cyclization of 2-alkynyltrifluoroacetanilides with aryl halides and vinyl triflates. Tetrahedron. 1994, 50, 437-52.
(3) Bai, R.; Schwartz, R. E.; Kepler, J. A.; Pettit, G. R.; Hamel, E., Characterization of the Interaction of Cryptophycin with Tubulin: Binding in the Vinca Domain, Competitive Inhibition of Dolastatin 10 Binding, and an Unusual Aggregation Reaction, Cancer Res., 1996, 56, 4398-4406.
(4) Boger, D. L.; Curran, T. T., Synthesis of the Lower Subunit of Rhizoxin, J. Org. Chem. 1992, 57, 2235.
(5a) Boyd, M. R.; Paull, K. D., Some Practical Considerations and Applications of the National Cancer Institute In Vitro Anticancer Drug Discovery Screen, Drug Development Research, 1995, 34, 91.
(5b) Bunker, A. M., Edmunds, J. J., Berryman, K. A., Walker, D. M., Flynn, M. A., Welch, K. M. and Doherty, A. M.; 1,3-Diaryl-2-carboxyindoles as Potent Non-peptide Endothelin Antagonists, Bioorg. Med. Chem. Lett., 1996, 6(9), 1061-1066.
(6) Chaplin, D. J.; Dougherty, G. J. Tumor vasculature as a target for cancer therapy. Br. J. Cancer Therapy. 1999, 80 (Suppl. 1), 57-64.
(7) Chaplin D J, Pettit G R, Hill S A. Anti-vascular approaches to solid tumor therapy: Evaluation of combretastatin A4 phosphate. Anticancer Res., 1999; 19:189-196.
(8) Chaplin J H, Flynn B L. A multi-component coupling approach to benzo[b]furans and indoles. Chem. Commun. 2001, 1594-5.
(9) Chavan, A. J.; Richardson, S. K.; Kim, H.; Haley, B. E.; Watt, D. S., Forskolin Photoaffinity Probes for the Evaluation of Tubulin Binding Sites, Bioconjugate Chem. 1993, 4, 268.
(10) Cortese, F.; Bhattacharyya, B.; Wolff, J., Podophyllotoxin as a Probe for the Colchicine Binding Site of Tubulin, J. Biol. Chem., 1977, 252, 1134.
(11) Cushman, M.; He, H.-M.; Katzenellenbogen, J. A.; Varma, R. K.; Hamel, E.; Lin, C. M.; Ram, S.; Sachdeva, Y. P., Synthesis of Analogs of 2-Methoxyestradiol with Enhanced Inhibitory Effects on Tubulin Polymerization and Cancer Cell Growth, J. Med. Chem., 1997.
(12) Cushman, M.; He, H-M.; Katzenellenbogen, J. A.; Lin, C. M.; Hamel, E., Synthesis, Antitubulin and Antimitotic Activity, and Cytotoxicity of Analogs of 2-Methoxyestradiol, an Endogenous Mammalian Metabolite of Estradiol That Inhibits Tubulin Polymerization by Binding to the Colchicine Binding Site, J. Med Chem., 1995, 38, 2041.
(13) Cushman, M.; Nagarathnam, D.; Gopal, D.; Chakraborti, A. K.; Lin, C. M.; Hamel, E. Synthesis and Evaluation of Stilbene and Dihydrostilbene Derivatives as Potential Anticancer Agents That Inhibit Tubulin Polymerization, J. Med Chem. 1991, 34, 2579.
(14) D'Amato, R. J.; Lin, C. M.; Flynn, E.; Folkman, J.; Hamel, E., 2Methoxyestradiol, an endogenous mammalian metabolite, inhibits tubulin polymerization by interacting at the colchicine site, Proc. Natl. Acad Sci. 1994, 91, 3964.
(15) Dark, G. G., Hill, S. A., Prise, V. G., Tozer, G. M., Pettit, G. R., Chaplin, D. J., Combretastatin A-4, an Agent That Displays Potent and Selective Toxicity Toward Tumor Vasculature, Cancer Res., 1997, 57, 1829-1834
(16) Davis P D, Dougherty G J, Blakey D C, Galbraith S M, Tozer G M, Holder A L, Naylor M A, Nolan J, Stratford M R, Chaplin D J, Hill S A. ZD6126: A Novel Vascular-targeting Agent that causes selective destruction of tumor vasculature. Cancer Research. 2002. 62(24): 7247-53.
(17) Dorr, R. T.; Dvorakova, K.; Snead, K.; Alberts, D. S.; Salmon, S. E.; Pettit, G. R., Antitumor Activity of Combretastatin A4 Phosphate, a Natural Product Tubulin Inhibitor, Invest. New Drugs, 1996, 14, 131.
(18) Fischer, B.; and Sheihet, L., J. Org. Chem. 1998, 63, 393-395.
(19) Floyd. L. J.; Bames, L. D.; Williams, R. F., Photoaffinity Labeling of Tubulin with (2-Nitro-4-azidophenyl)deacetylcolchicine: Direct Evidence for Two Colchicine Binding Sites, Biochemistry, 1989, 28, 8515.
(20) Flynn B L, Hamel E, Junk M K. One-pot synthesis of Benzo[b]furan and Indole inhibitors of Tubulin polymerization. J. Med. Chem. 2002, 45: 2670-3.
(21) Gastpar, R.; Goldbrunner, M.; Marko, D.; von Angerer, E., Methoxy Substituted 3-Formyl-2-phenylindoles Inhibit Tubulin Polymerization, J. Med Chem. 1998, 41, 4965-4972.
(22) Gerwick, W. H.; Proteau, P. J.; Nagle, D. G.; Hamel, B.; Blokhin, A.; Slate, D. L., Structure of Curacin A, a Novel Antimitotic, Antiproliferative, and Brine Shrimp Toxic Natural Product from the Marine Cyanobacterium Lyngbya majuscula, J. Org. Chem. 1994, 59, 1243.
(23) Gianna Kakou, P.; Sackett, D.; Fojo, T.; Tubulin/Microtubes: Still a promising Target for New Chemotherapeutic Agents, J. Natl Cancer Inst., 2000, 92, 182.
(24) Grese, T. A.; Cho, S.; Finley, D. R.; Godfrey, A. G.; Jones, C. D.; Lugar m, C. W.; Martin, M. J.; Matsumoto, K; Pennington, L. D.; Winter, M. A.; Adrian, M. D.; Cole, H. W.; Magee, D. E.; Phillips, D. L.; Rowley, E. R.; Short, L.; Glasebrook, A. L.; Bryant, H. R., Structure-Activity Relationships of Selective Estrogen Receptor Modulators: Modifications to the 2-Arylbenzothiophene Core of Raloxifene, J Med Chem., 1997, 40, 146.
(25) Hadimani, M. B.; Kessler, R. J.; Kautz, J. A.; Ghatak, A.; Shirali, A. R.; O'Dell, H.; Garner, C. M.; Pinney, K. G. 2-(3-tert-Butyldimethylsiloxy-4-methoxyphenyl)-6-methoxy-3-(3,4,5-trimethoxybenzoyl)indole. Acta Cryst. 2002, C58, o330-o332.
(26) Hahn, K. M.; Hastie, S. B.; Sundberg, R. J., Synthesis and Evaluation of 2-Diazo-3,3,3-trifluoropropanoyl Derivatives of Colchicine and Podophyllotoxin as Photoaffinity Labels: Reactivity, Photochemistry, and Tubulin Binding, Photochem. Photobiol. 1992, 55, 17.
(27) Hamel, E.; Lin, C. M.; Flynn, E.; D'Amato, R. J. D., Interactions of 2-Methoxyestradiol, and Endogenous Mammalian Metabolite, with Unpolymerized Tubulin and with Tubulin Polymers, Biochemistry, 1996, 35, 1304.
(28) Hamel, E., Antimitotic Natural Products and Their Interactions with Tubulin, Medicinal Research Reviews, 1996, 16, 207.
(29) Hamel, E.; Lin, C. M., Separation of Active Tubulin and Microtubule-Associated Proteins by Ultracentrifugation and Isolation of a Component Causing the Formation of Microtubule Bundles, Biochemistry, 1984, 23, 4173-4184.
(30) Hamel, E.; Lin, C. M. Interactions of combretastatin, a new plant-derived antimitotic agent, with tubulin. Biochem. Pharmacol. 1983, 32, 3864-3867.
(31) Hammonds, T. R.; Denyer, S. P.; Jackson, D. B.; Irving, W. L., Studies To Show That With Podophyllotoxin the Early Replicative Stages of Herpes Simplex Virus Type 1 Depend Upon Functional Cytoplasmic Microtubules, J. Med. Microbiol., 1996, 45, 167.
(32) Inion, H.; De Vogelaer, H.; Descamps, M.; Bauthier, J.; Colot, M.; Richard, J; Charlier, R. Study of the indole series. IV. 2-Alkyl or 2-aryl 3-(4-aminoalkoxybenzoyl)indoles as potential antianginal and antiinflammatory compounds. Eur. J. Med. Chem. 1977, 12, 483-487.
(33) Iyer, S.; Chaplin, D. J.; Rosenthal, D. S.; Boulares, A. H.; Li, L. Y.; and Smulson, M. E., Cancer Res. 1998, 58, 4510-4514.
(34) Jiang, J. B.; Hesson, D. P.; Dusak, B. A.; Dexter, D. L.; Kang, G. J.; Hamel, B., Synthesis and Biological Evaluation of 2-Styrylquinazolin-4(3H)-ones, a New Class of Antimitotic Anticancer Agents Which Inhibit Tubulin Polymerization, J. Med. Chem. 1990, 33, 1721.
(35) Jones, C. K.; Jevnikar, M. G.; Pike, A. J.; Peters, M. K.; Black, L. J.;Thompson, A. R.; Falcone, J. F.; Clemens, J. A., Antiestrogens. 2. Structure-Activity Studies in a Series of 3-Aroyl-2-arylbenzo[b]thiophene Derivatives Leading to [6-Hydroxy-2-(4-hydroxyphenyl) benzo[b]thiphene-3-yl][4-[2-(1-piperidinyl)ethoxy] phenylimethanone Hydrochloride (LY156758), a Remarkably Effective Estrogen Antagonist with Only Minimal Intrinsic Estrogenicity, J. Med Chem. 1984, 27, 1057.
(36) Kanthou C, Tozer G M. The tumor vascular targeting agent CA4P induces reorganization of the actin cytoskeleton and early membrane blebbing in human endothelial cells. Blood. 2002. 99(6):2060-9.
(37) Kingston, D. G. I.; Samaranayake, G.; Ivey, C. A., The Chemistry of Taxol, a Clinically Useful Anticancer Agent, J. Nat. Prod. 1990, 53, 1.
(38) Kobayashi, S.; Nakada, M.; Ohno, M., Synthetic Study on an Antitumor Antibiotic Rhizoxin by Using an Enzymatic Process on Prochiral beta-substituted Glutarates, Pure Appl. Chem. 1992, 64, 1121.
(39) Kobayashi, S.; Nakada, M.; Ohno, M., Synthetic Study on an Antitumor Antibiotic Rhizoxin by Using an Enzymatic Process on Prochiral beta-substituted Glutarates Indian J. Chem., Sect. B. 1993, 32B, 159.
(40) Lavielle, G.; Havtefaye, P.; Schaeffer, C.; Boutin, J. A.; Cudennec, C. A.; Pierre, A., New α-Amino Phosphonic Acid Derivatives of Vinblastine: Chemistry and Antitumor Activity, J Med Chem. 1991, 34, 1998.
(41) Lejeune P, Hodge T G, Vrignaud, Bissery M-C. In vivo antitumor activity and tumor necrosis induced by AVE8062A, a tumor vasculature targeting agent. Proceedings of the AACR. ABSTRACT#781. 2002, 43: 156.
(42a) Lin, C. M.; Ho, H. H.; Pettit, G. R.; Hamel, E., Antimitotic Natural Products Combretastatin A-4 and Combretastatin A-2: Studies on the Mechanism of Their Inhibition of the Binding of Colchicine to Tubulin, Biochemistry 1989, 28, 6984.
(42b) Liu, Y and McWhorter, Jr., W. W; Synthesis of 8-Desbromohinckdentine A, J. Am. Chem. Soc., 2003, 125, 4240-4252.
(42c) Liu, Y. and Gribble, G. W.; Bis-Suzuki reactions of 2,3-dihaloindoles. A convenient synthesis of 2,3-diarylindoles, Tetrahedron Letters, 2000, 41, 8717-8721.
(42d) Masanobu, T., Hiroyo, I., Mayumi, T., Tetsuji, H., Hiroko, T., Chiaki, K., Toshiko, N., Mikiko, S. and Masahiko, U.; Synthetic studies of indoles and related compounds: Regioselective bromination of methoxy derivatives of ethyl indole-2-carboxylate, Heterocycles, 1992, 34(12), 2349-2362. (43) Monks, A.; Scudiero, D.; Skehan, P.; Shoemaker, R.; Paull, K.; Vistica, D.; Hose, C.; Langley, J.; Cronise, P.; Vaigro-Wolff, A.; Gray-Goodrich; Campbell; Mayo; Boyd, M., Feasibility of a High-Flux Anticancer Drug Screen Using a Diverse Panel of Cultured Human Tumor Cell Lines, J. Natl. Cancer Inst., 1991, 83, 757-766.
(44) Mossman, T. Rapid colorimetric assay for cellular growth and survival: application to proliferation and cytotoxicity assay. J. Immunol. Methods, 1983, 16, 195-200.
(45) Mullica, D. F.; Pinney, K. G.; Mocharla, V. P.; Dingeman, K. M.; Bounds, A. D.; Sappenfield, E. L., Characterization and Structural Analyses of Trimethoxy and Triethoxybenzo[b]thiophene, J. Chem. Cryst., 1998, 28, 289-295.
(46) Nakada, M.; Kobayashi, S.; Iwasaki, S.; Ohno, M., The First Total Synthesis of the Antitumor Macrolide Rhizoxin: Synthesis of the Key Building Blocks, Tetrahedron Lett 1993, 34, 1035.
(47) Nicolaou, K. C., Winssinger, N., Pastor, J., Ninkovic, S., Sarabia, F., He, Y., Vourloumis, D., Yang, Z., Oi, T., Giannakakou, P., Hamel, B., Synthesis of Epothilones A and B in Solid and Solution Phase, Nature, 1997, 387, 268-272.
(48) Owellen, R. J.; Hartke, C. A.; Kickerson, R. M.; Hams, F. 0., Inhibition of Tubulin-Microtubule Polymerization by Drugs of the Vinca Alkaloid Class, Cancer Res. 1976, 36, 1499.
(49) Palkowitz, A. D.; Glasebrook, A. L.; Thrasher, K. J.; Hauser, K. L.; Short, L. L.; Phillips, D. L.; Muehl, B. S.; Sato, M.; Shetler, P. K.; Cullinan, G. J.; Pell, T. R.; Bryant, H. U., Discovery and Synthesis of [6-Hydroxy-3-[4-[2-(1-piperidinyl)ethoxy]phenoxy]-2-(4-hydroxyphenyl)] benzo[b]thiophene: A Novel, Highly Potent, Selective Estrogen Receptor Modulator, J. Med Chem., 1997, 40, 1407.
(50) Parness, J.; Horwitz, S. B., Taxol Binds to Polymerized Tubulin In Vitro, J. Cell Biol. 1981, 91, 479.
(51) Pettit, G. R.; and Rhodes, M. R. Antineoplastic agents 393. Synthesis of the trans-isomer of CA4P. Anti-Cancer Drug Des. 1998, 13, 183-191.
(52) Pettit, G. R., Toki, B., Herald, D. L., Verdier-Pinard, P., Boyd, M. R., Hamel, E., Pettit, R. K., Antineoplastic Agents 379. Synthesis of Phenstatin Phosphate, J. Med Chem., 1998, 41, 1688-1695.
(53) Pettit, G. R., Srirangam, J. K., Barkoczy, J., Williams, M. D., Boyd, M. R., Hamel, E., Pettit, R. K, Hogan F., Bai, R., Chapuis, J. C., McAllister, S. C., Schmidt, J. M., Antineoplastic Agents 365: Dolastatin 10 SAR Probes, Anti-Cancer Drug Des., 1998, 13, 243-277.
(54) Pettit, G. R.; Cragg, G. M.; Singh, S. B., Antineoplastic agents, 122. Constituents of Combretum caffrum, J. Nat. Prod 1987, 50, 386.
(55) Pettit, G. R., Kamano, Y., Herald, C. L., Tuinman, A. A., Boettner, F. E., Kizu, H., Schmidt, J. M., Baczynskyj, L., Tomer, K. B., Bontems, R. J., The Isolation and Structure of a Remarkable Marine Animal Antineoplastic Constituent: Dolastatin 10, J. Am. Chem. Soc., 1987, 109, 6883-6885.
(56) Pettit, G. R.; Singh, S. B.; Cragg, G. M., Synthesis of Natural (-)Combretastatin, J. Org. Chem. 1985, 50, 3404.
(57) Pettit, G. R.; Cragg, G. M.; Herald, D. L.; Schmidt, J. M.; Lohavanijaya, P., Isolation and Structure of combretastatin, Can. J. Chem. 1982, 60, 1374.
(58) Pinney et al., Synthesis of a Benzo[b]thiophene-based Vascular Targeting Prodrug and Related Anti-Tubulin Ligands, 220th American Chemical Society, National Meeting, Division of Organic Chemistry, Abstract No. 196, Washington, D.C., Aug. 20-24, 2000.
(59) Pinney, K. G.; Dingeman, K. D.; Bounds, A. D.; Mocharla, V. P.; Pettit, G. R.; Bai, R.; Hamel, E., A New Anti-Tubulin Agent Containing the Benzo[b]thiophene Ring System, Bioorganic and Medicinal Chemistry Letters, 1999, 9, 1081-1086.
(60) Rao, A. V. R.; Bhanu, M. N.; Sharma, G. V. M., Studies Directed Towards the Total Synthesis of Rhizoxin: Stereoselective Synthesis of C-12 to C-18 Segment, Tetrahedron Lett. 1993, 34, 707.
(61) Rao, A. V. R.; Sharma, G. V. M.; Bhanu, M. N., Radical Mediated Enantioselective Construction of C-1 to C-9 Segment of Rhizoxin, Tetrahedron Lett. 1992, 33, 3907.
(62) Rao, S.; Horwitz, S. B.; Ringel, I., Direct Photoaffinity Labeling of Tubulin with Taxol, J. Natl. Cancer Inst., 1992, 84, 785.
(63) Sawada, T.; Kato, Y.; Kobayashi, H.; Hashimoto, Y.; Watanabe, T.; Sugiyama, Y.; Iwasaki, S., A Fluorescent Probe and a Photoaffinity Labeling Reagent to Study the Binding Site of Maytansine and Rhizoxin on Tubulin, Bioconjugate Chem., 1993, 4, 284.
(64) Sawada, T.; Kobayashi, H.; Hashiinoto, Y.; Iwasaki, S., Identification of the Fragment Photoaffinity-labeled with Azidodansyl-rhizoxin as Met-363-Lys-379 on beta-Tubulin, Biochem. Pharmacol. 1993, 45, 1387.
(65) Sawada, T.; Hashimoto, Y.; Li, Y.; Kobayashi, H.; Iwasaki, S., Fluorescent and Photoaffinity Labeling Derivatives of Rhizoxin, Biochem. Biophys. Res. Commun. 1991, 178, 558.
(66) Safa, A. R.; Hamel, E.; Felsted, R. L., Photoaffinity Labeling of Tubulin Subunits with a Photoactive Analog of Vinblastine, Biochemistry 1987, 26, 97.
(67) Schiff, P. B.; Fant, J.; Horwitz, S. B., Promotion of Microtubule Assembly In Vitro by Taxol, Nature, 1979, 277, 665.
(68) Staretz, M. E.; Hastie, S. B., Synthesis, Photochemical Reactions, and Tubulin Binding of Novel Photoaffinity Labeling Derivatives of Colchicine, J. Org. Chem. 1993, 58, 1589.
(69) Swindell, C. S.; Krauss, N. B.; Horwitz, S. B.; Ringel, I., Biologically Active Taxol Analogs with Deleted A-ring Side Chain Substituents and Variable C-2,AE Configurations, J. Med Chem. 1991, 34, 1176.
(70) Taylor, S. D.; Chen, M. J,; Dinaut, A. N.; and Batey, R. A., Tetrahedron 1998, 54, 4223-4242.
(71) Tozer, G. M.; Prise, V. E.; Wilson, J.; Locke, R. J.; Vojnovic, B.; Stratford, M. R. L.; Dennis, M. F.; Chaplin, D. J., Combretastatin A-4 Phosphate as a Tumor Vascular-Targeting Agent: Early Effects in Tumors and Normal Tissues. Cancer Res., 1999, 59, 1626.
(72) Von Angerer, E. Tubulin as a target for anticancer drugs. Current opinion in Drug Discovery and Development. 2000, 3, 575-584.
(73) Von Angerer, E.; Prekajac, J.; Strohmeier, J. 2-Phenylindoles. Relationship between Structure, Estrogen Receptor Affinity, and Mammary Tumor Inhibiting Activity in the Rat. J. Med. Chem. 1984, 27, 1439-1447.
(74) Williams, R. F.; Mumford, C. L.; Williams, G. A.; Floyd, L. J.; Aivaliotis, M. J.; Martinez, R. A.; Robinson, A. K.; Bames, L. D., A Photoaffinity Derivative of Colchicine: 6-(4′-Azido-2′-nitrophenylamino)hexanoyldeacetylcolchicine. Photolabeling and Location of the Colchicine-binding Site on the alpha-subunit of Tubulin, J Biol. Chem. 1985, 260, 13794.
(75) Wolff, J.; j(nipling. L.; Cahumann, H. J.; Palumbo, G., Direct Photoaffinity Labeling of Tubulin with Colchicine, Proc. Natl. Acad Sci. U.S.A. 1991, 88, 2820.
(76) Zhang, X.; Smith, C. D., Microtubule Effects of Welwistatin, a Cyanobacterial Indolinone that Circumvents Multiple Drug Resistance, Molecular Pharmacology, 1996, 49, 288.

1. A compound of formula (I): 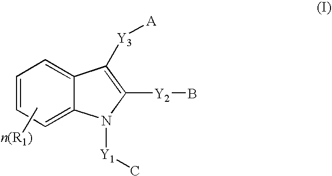
wherein
R₁ is independently selected from the group consisting of OH, nitro, amine, lower alkyl, lower alkoxy, phosphate, or halogen;
n is 0, 1, 2, 3, or 4;
Y₁, Y₂, are Y₃ are selected from the group consisting of a covalent bond, —CO—, —O—, —S—, —CH₂—, or —CH₂O—; and
A, B, and C are independently alkyl, aryl or H,
wherein at least two of A, B, or C are aryl; or a prodrug thereof.

2. The compound of claim 1, wherein n is 1 or 2.

3. The compound of claim 1, wherein R₁ is a lower alkoxy group.

4. The compound of claim 1, wherein Y₃ is a carbonyl group, A is a trisubstituted aryl group, B is a di- or tri-substituted aryl group, Y₂ is a covalent bond, Y₁ is a covalent bond, and C is H.

5. The compound of claim 1, wherein Y₃ is a covalent bond, A is a di- or tri-substituted aryl group, B is a trisubstituted aryl group, Y₂ is a carbonyl group, Y₁ is a covalent bond, and C is H.

6. The compound of claim 1, wherein Y₃ is a covalent bond, A is H, B is a di- or tri-substituted aryl group, Y₂ is a covalent bond, Y₁ is a carbonyl group, and C is a trisubstituted aryl group.

7. A method for inhibiting tubulin polymerization in vitro by contacting a cell with an effective amount of a compound of claim 1.

8. The method of claim 7, wherein said cell is a tumor cell.

9. A method of treating a mammal afflicted with a neoplastic disease by administering to said mammal a therapeutically effective amount of a compound of claim 1.

10. The method of claim 7, wherein the contacted cell is located in a patient.

11. A method for treating cancer by administering to a patient in need thereof, a therapeutically effective amount of a compound of claim 1, wherein said cancer is selected from the group consisting of leukemia, lung cancer, colon cancer, thyroid cancer, CNS cancer, melanoma, ovarian cancer, renal cancer, prostate cancer, pancreatic cancer, and breast cancer.

12. A pharmaceutical composition comprising a compound of claim 1 as an active component along with a pharmaceutically acceptable carrier.

13. A method for selectively destroying tumor vasculature in a patient comprising administering an effective amount of a compound of claim 1.

14. A method for selectively reducing blood flow to at least a portion of a neoplastic region, comprising administering an effective amount of a compound of claim 1, wherein substantial necrosis of tissue in the neoplastic region without substantial necrosis of tissue in adjoining regions is effected.

15. The method of claim 14, wherein the effect of reduced tumor blood flow is reversible.
